SEQUENCE LISTING (8 sequences)

SEQ ID NO: 1 - DNA, 21 bp, Homo sapiens
taccagggcgaagtggggagg

SEQ ID NO: 2 - DNA, 21 bp, Homo sapiens
cacctgtgccattctggtggg

SEQ ID NO: 3 - DNA, 29 bp, Homo sapiens
aataggtgtcgatttgcagtgacaatgtg

SEQ ID NO: 4 - DNA, 29 bp, Homo sapiens
ccaagtgactgacaatctggtctaacgaa

SEQ ID NO: 5 - DNA, 20 bp, Homo sapiens
tccttctgtttcatccaagc

SEQ ID NO: 6 - DNA, 20 bp, Homo sapiens
tactagcggttttacgggcg

SEQ ID NO: 7 - DNA, 21 bp, Homo sapiens
agcatctacagtgtcatgggg

SEQ ID NO: 8 - DNA, 22 bp, Homo sapiens
gattcaaatttgggggctgcct

(19) United States
(12) Patent Application Publication
(10) Pub. No.: US 2023/0057542 A1
(43) Pub. Date: Feb. 23, 2023

(54) METHODS AND MATERIALS FOR ASSESSING AND TREATING CMV INFECTIONS

(71) Applicant: University of Pittsburgh - Of the Commonwealth System of Higher Education, Pittsburgh, PA (US)

(72) Inventors: John F. McDyer, Pittsburgh, PA (US); Iulia-Dana Popescu, Pittsburgh, PA (US); Akihiko Hoji, Carmel, IN (US)

(21) Appl. No.: 17/623,761

(22) PCT Filed: Jul. 6, 2020
(86) PCT No.: PCT/US2020/040913

§ 371 Date: Dec. 29, 2021

Related U.S. Application Data
(60) Provisional application No. 62/870,495, filed on Jul. 3, 2019.

(51) Int. Cl. A61K 31/675; A61K 31/522; A61K 31/662; A61P 31/20

(52) U.S. Cl. CPC A61K 31/675; A61K 31/522; A61K 31/662; A61P 31/20

(57) ABSTRACT

This document relates to methods and materials involved in identifying and/or treating mammals having a CMV infection. For example, methods and materials for assessing a mammal having a CMV infection (e.g., a CMV seronegative human who received CMV seropositive donor transplant tissue) to determine if the mammal is likely to control the CMV infection or to determine if the mammal is unlikely to control the CMV infection are provided. Methods and materials for treating a mammal having a CMV infection (e.g., a CMV seronegative human who received CMV seropositive donor transplant tissue) that was either identified as being likely to control the CMV infection or identified as being unlikely to control the CMV infection also are provided.

### CROSS-REFERENCE TO RELATED APPLICATION

This application is a National Stage application under 35 U.S.C. § 371 of International Application No. PCT/US2020/040913, having an International Filing Date of Jul. 6, 2020, which claims the benefit of U.S. patent application Ser. No. 62/870,495, filed on Jul. 3, 2019. The disclosures of the prior applications are considered part of (and are incorporated by reference in) the disclosure of this application.

### STATEMENT AS TO FEDERALLY SPONSORED RESEARCH

This invention was made with government support under AI079175 awarded by National Institutes of Health. The government has certain rights in the invention.

### SEQUENCE LISTING

This application contains a Sequence Listing that has been submitted electronically as an ASCII text file named 45049_0025WO1_ST25.txt. The ASCII text file, created on Jun. 25, 2020, is 2 kilobytes in size. The material in the ASCII text file is hereby incorporated by reference in its entirety

### BACKGROUND

### 1. Technical Field

This document relates to methods and materials involved in assessing and/or treating mammals having a cytomegalovirus (CMV) infection. For example, this document provides methods and materials for assessing a mammal having a CMV infection (e.g., a CMV seronegative human who received CMV seropositive donor transplant tissue) to determine if the mammal is highly likely to control the CMV infection, to determine if the mammal is likely to control the CMV infection, or to determine if the mammal is unlikely to control the CMV infection. This document also provides methods and materials for appropriately treating mammals having a CMV infection (e.g., a CMV seronegative human who received CMV seropositive donor transplant tissue) that are identified as being highly likely, likely, or unlikely to control the CMV infection.

### 2. Background Information

CMV can cause a chronic viral infection and is an opportunistic pathogen in solid organ transplant recipients (SOTRs) as well as hematopoietic cell transplant recipients. Patients (e.g., a lung transplant recipients (LTRs)) that are seronegative but receiving seropositive donor tissue have increased risk for CMV complications (Fishman, N. Engl. J. Med., 357:2601-14 (2007)). Risk of recurrent CMV reactivation in SOTRs remains a problem given the adverse side effects of antiviral therapy especially the effects associated with prolonged exposure to antiviral agents (Teira et al., Blood, 127:2427-28 (2016)). Thus, there remains an unmet need in the management of CMV infections.

### SUMMARY

This document provides methods and materials involved in assessing and/or treating mammals (e.g., humans) having a CMV infection. For example, this document provides methods and materials for assessing a mammal having a CMV infection (e.g., a CMV seronegative human who received CMV seropositive donor transplant tissue) to determine if the mammal is highly likely to control the CMV infection, to determine if the mammal is likely to control the CMV infection, or to determine if the mammal is unlikely to control the CMV infection.

As described herein, humans infected with CMV (e.g., a CMV seronegative human who received CMV seropositive donor transplant tissue) identified as having a highly elevated level of KLRG1⁺/CD8⁺ T cells can be classified as being highly likely to control the CMV infection, humans infected with CMV identified as having a moderately elevated level of KLRG1⁺/CD8⁺ T cells can be classified as being likely to control the CMV infection, and humans infected with CMV identified as lacking an elevated level of KLRG1⁺/CD8⁺ T cells can be classified as being unlikely to control the CMV infection. Having the ability to classify mammals infected with CMV as being highly likely, likely, or unlikely to control the CMV infection as described herein can allow clinicians and patients to proceed with appropriate CMV infection treatment options. For example, having the ability to classify mammals infected with CMV as being highly likely to control the CMV infection without further medical intervention can allow clinicians and patients to avoid unnecessary anti-viral treatment options that may cause adverse side effects, while having the ability to classify mammals infected with CMV as being unlikely to control the CMV infection without further medical intervention can allow clinicians and patients to proceed with necessary anti-viral treatment options despite possible adverse side effects.

This document also provides methods and materials for treating a mammal having a CMV infection (e.g., a CMV seronegative human who received CMV seropositive donor transplant tissue) that was identified as being highly likely to control the CMV infection, identified as being likely to control the CMV infection, or identified as being unlikely to control the CMV infection.

As described herein, humans infected with CMV (e.g., a CMV seronegative human who received CMV seropositive donor transplant tissue) who were identified as having a highly elevated level of KLRG1⁺/CD8⁺ T cells can be occasionally monitored to confirm that no antiviral therapy is needed to control the CMV infection, while humans infected with CMV (e.g., a CMV seronegative human who received CMV seropositive donor transplant tissue) who were identified as having a moderately elevated level of KLRG1⁺/CD8⁺ T cells can be monitored (e.g., regularly monitored at least once every 4 to 12 weeks) and administered one or more antiviral agents when active CMV infection is detected, and humans infected with CMV who were identified as lacking an elevated level of KLRG1⁺/CD8⁺ T cells can be treated in a manner that involves, for example, prolonged use of antiviral agents to control the CMV infection. Having the ability to treat mammals infected with CMV that were identified as being highly likely, likely, or unlikely to control the CMV infection as described herein can allow clinicians and patients to proceed confidently with appropriate CMV infection treatment options. For example, having the ability to monitor mammals infected with CMV that were identified as being likely to control the CMV infection in a manner that avoids the unnecessary use of anti-viral agents can allow clinicians and patients to avoid adverse side effects from such use of anti-viral agents, while having the ability to treat mammals infected with CMV that were identified as being unlikely to control the CMV infection without further medical intervention can allow clinicians and patients to proceed confidently with necessary anti-viral treatment options despite possible adverse side effects.

In general, one aspect of this document features methods for identifying a mammal as being highly likely to control a CMV infection, where the methods can include, or can consist essentially of, (a) determining that the mammal contains a highly elevated level of KLRG1⁺/CD8⁺ T cells, and (b) classifying the mammal as being highly likely to control the CMV infection. The mammal can be a human. The mammal can be a recipient of a transplant. The transplant can be positive for the CMV infection. The mammal can be a donor positive/recipient negative mammal for the CMV infection or a donor negative/recipient positive mammal for the CMV infection. The CMV infection can be a pre-acute, acute/primary, contraction phase, or chronic CMV infection. The highly elevated level can be a level where more than 50 percent of CD8⁺ T cells are KLRG1⁺/CD8⁺ T cells. The highly elevated level can be a level where more than 55 percent of CD8⁺ T cells are KLRG1⁺/CD8⁺ T cells. The method also can include monitoring the CMV infection within said mammal. The method can include avoiding the administration of an antiviral agent to treat the mammal. The method also can include administering an immunosuppressant to the mammal. The immunosuppressant can be tacrolimus, prednisone, everolimus, mycophenolate mofetil, or azathioprine.

In another aspect, this document features methods for identifying a mammal as being likely to control a CMV infection where the methods can include, or can consist essentially of, (a) determining that the mammal contains a moderately elevated level of KLRG1⁺/CD8⁺ T cells, and (b) classifying the mammal as being likely to control the CMV infection. The mammal can be a human. The mammal can be a recipient of a transplant. The transplant can be positive for the CMV infection. The mammal can be a donor positive/recipient negative mammal for the CMV infection or a donor negative/recipient positive mammal for the CMV infection. The CMV infection can be a pre-acute, acute/primary, contraction phase, or chronic CMV infection. The moderately elevated level can be a level wherein more than 40 percent and less than or equal to 50 percent of CD8⁺ T cells are KLRG1⁺/CD8⁺ T cells. The highly elevated level can be a level wherein more than 43 percent and less than or equal to 48 percent of CD8⁺ T cells are KLRG1⁺/CD8⁺ T cells. The method also can include monitoring the CMV infection within the mammal. The method can include avoiding the administration of an antiviral agent to treat the mammal. The method also can include administering an immunosuppressant to the mammal. The immunosuppressant can be tacrolimus, prednisone, everolimus, mycophenolate mofetil, or azathioprine.

In another aspect, this document features methods for identifying a mammal as being unlikely to control a CMV infection, where the methods can include, or can consist essentially of, (a) determining that the mammal lacks an elevated level of KLRG1⁺/CD8⁺ T cells, and (b) classifying the mammal as being unlikely to control the CMV infection. The mammal can be a human. The mammal can be a recipient of a transplant. The transplant can be positive for the CMV infection. The mammal can be a donor positive/recipient negative mammal for the CMV infection or a donor negative/recipient positive mammal for the CMV infection. The CMV infection can be a pre-acute, acute/primary, contraction phase, or chronic CMV infection. The mammal can have a level of KLRG1⁺/CD8⁺ T cells where less than 40 percent of CD8⁺ T cells are KLRG1⁺/CD8⁺ T cells. The mammal can have a level of KLRG1⁺/CD8⁺ T cells where less than 35 percent of CD8⁺ T cells are KLRG1⁺/CD8⁺ T cells. The method also can include monitoring the CMV infection within the mammal. The method also can include administering an antiviral agent to the mammal. The antiviral agent can be ganciclovir, valganciclovir, foscarnet, or cidofovir. The method also can include administering an immunosuppressant to the mammal. The immunosuppressant can be tacrolimus, prednisone, everolimus, mycophenolate mofetil, or azathioprine.

In another aspect, this document features methods for treating a CMV infection, where the methods can include, or consist essentially of, administering, to a mammal (a) having the CMV infection and (b) identified as lacking an elevated level of KLRG1⁺/CD8⁺ T cells, an antiviral agent. The mammal can be a human. The mammal can be a recipient of a transplant. The transplant can be positive for the CMV infection. The mammal can be a donor positive/recipient negative mammal for the CMV infection or a donor negative/recipient positive mammal for the CMV infection. The CMV infection can be a pre-acute, acute/primary, contraction phase, or chronic CMV infection. The mammal can have a level of KLRG1⁺/CD8⁺ T cells where less than 40 percent of CD8⁺ T cells are KLRG1⁺/CD8⁺ T cells. The mammal can have a level of KLRG1⁺/CD8⁺ T cells where less than 35 percent of CD8⁺ T cells are KLRG1⁺/CD8⁺ T cells. The antiviral agent can be ganciclovir, valganciclovir, foscarnet, or cidofovir. The antiviral agent can be administered until a CMV copy number is less than 300 CMV copies per mL of blood within the mammal. The method also can include administering an immunosuppressant to the mammal. The immunosuppressant can be tacrolimus, prednisone, everolimus, mycophenolate mofetil, or azathioprine.

In another aspect, this document features methods for treating a CMV infection, where the methods can include, or consist essentially of, (a) administering an antiviral agent to a mammal having the CMV infection if the mammal was identified as lacking an elevated level of KLRG1⁺/CD8⁺ T cells, and (b) monitoring the mammal for an uncontrolled CMV infection if the mammal was identified as having a moderately elevated level or a highly elevated level of KLRG1⁺/CD8⁺ T cells. The mammal can be a human. The mammal can be a recipient of a transplant. The transplant can be positive for the CMV infection. The mammal can be a donor positive/recipient negative mammal for the CMV infection or a donor negative/recipient positive mammal for the CMV infection. The CMV infection can be a pre-acute, acute/primary, contraction phase, or chronic CMV infection. The elevated level can be a level where more than 40 percent of CD8⁺ T cells are KLRG1⁺/CD8⁺ T cells. The elevated level can be a level where more than 45 percent of CD8⁺ T cells are KLRG1⁺/CD8⁺ T cells. The moderately elevated level can be a level where more than 40 percent and less than or equal to 50 percent of CD8⁺ T cells are KLRG1⁺/CD8⁺ T cells. The highly elevated level can be a level wherein more than 50 percent of CD8⁺ T cells are KLRG1⁺/CD8⁺ T cells. The antiviral agent can be ganciclovir, valganciclovir, foscarnet, or cidofovir. The mammal can be identified as lacking the elevated level of KLRG1⁺/CD8⁺ T cells. The mammal can be identified as having the moderately elevated level of KLRG1⁺/CD8⁺ T cells. The mammal can be identified as having the highly elevated level of KLRG1⁺/CD8⁺ T cells. The method also can include administering an immunosuppressant to the mammal. The immunosuppressant can be tacrolimus, prednisone, everolimus, mycophenolate mofetil, or azathioprine.

In another aspect, this document features methods for monitoring a CMV infection in a manner that avoids the need to administer an antiviral agent to a mammal having the CMV infection, where the methods can include, or consist essentially of, assessing a symptom of the CMV infection in the mammal and avoiding administration of an antiviral agent to the mammal, where the mammal was identified as having a moderately elevated or highly elevated level of KLRG1⁺/CD8⁺ T cells. The mammal can be a human. The mammal can be a recipient of a transplant. The transplant can be positive for the CMV infection. The mammal can be a donor positive/recipient negative mammal for the CMV infection or a donor negative/recipient positive mammal for the CMV infection. The CMV infection can be a pre-acute, acute/primary, contraction phase, or chronic CMV infection. The moderately elevated level can be a level where more than 40 percent and less than or equal to 50 percent of CD8⁺ T cells are KLRG1⁺/CD8⁺ T cells. The highly elevated level can be a level where more than 50 percent of CD8⁺ T cells are KLRG1⁺/CD8⁺ T cells. The symptom can be CMV viremia or CMV positivity in BAL fluid. The mammal can be identified as having the moderately elevated level of KLRG1⁺/CD8⁺ T cells. The mammal can be identified as having the highly elevated level of KLRG1⁺/CD8⁺ T cells. The method also can include administering an immunosuppressant to the mammal. The immunosuppressant can be tacrolimus, prednisone, everolimus, mycophenolate mofetil, or azathioprine.

In another aspect, this document features a kit for determining the level of KLRG1⁺/CD8⁺ T cells present within a sample obtained from a mammal (e.g., a human). The kit comprises (or consists essentially of or consists of) an antibody that specifically binds to a KRLG polypeptide, an antibody that specifically binds to a CD8 polypeptide, and optionally an antibody that specifically binds to a CD3 polypeptide. The kit can comprise an antibody that specifically binds to a CD57 polypeptide. The kit can comprise a dead cell exclusion dye. The kit can comprise a package insert setting forth a cut off value for a moderately or highly elevated level of KLRG1⁺/CD8⁺ T cells.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A-1G. Rapid induction of KLRG1 and CD57 surface expression during primary CMV infection. A: Expression of CD57 and KLRG1 on CD8⁺ T cells based on flow cytometry of ex vivo stained PBMCs from Donor+/Recipient− (D⁺/R⁻) patients taken during pre-acute (pre-CMV) and acute/primary (acute CMV) infection. Total CD8⁺ T cells were gated on live CD3⁺ T cells, which were excluded from CD14⁺, CD16⁺, and CD19⁺ cells. The upper left quadrant represents CD8^{+/high} T cells expressing either CD57 or KLRG1. B: Distributions of CD57⁺ or KLRG1⁺ CD8⁺ T cells (n=23) represented in A. Line indicates mean±SEM. C: Expression profiles of total CD8⁺ T cells expressing CD57 or CD27. D: Expression profiles of gated CD8⁺ T cells expressing CD57 and KLRG1 and expression profiles of CD57⁺ and CD27⁺ on gated CD8⁺ T cells. PBMCs from a different D⁺/R⁻ patient with primary and chronic infection. E: Proportions of total CD8⁺ T cells expressing particular phenotypic markers from expression profiles in FIG. 1D. PBMCs from acute (n=15) and chronic (n=12) CMV infected D⁺/R⁻ patients. Acute samples represented by circles and chronic samples represented by squares. Lines match acute and chronic samples via a phenotypic marker. F: Gated on MHC Class I Dextramer⁺ positive cells, the plots show expression profiles for CD57 and CD27 (top row) and expression profiles for CD57 and KLRG1 expression (bottom row). PBMCs from the same patient as in FIG. 1D were stained with CMV pp65-specific Dextramers. G: Proportions of MHC Class I Dextramer⁺ cells from acute (n=14) and chronic (n=14) infection expressing the phenotypic markers from FIG. 1F.

FIG. 2A-2G. Preservation of CD57⁺ and KLRG1⁺ CMV-specific CD8⁺ T cells from primary to chronic CMV infection. A: Proliferation of CD57⁺ and KLRG1⁺ CMV pp65-specific CD8⁺ T cells from acute CMV infection. PBMCs were labeled with CFSE and stimulated in the presence of CMV pp65 peptide-mix for 7 days. Pseudo color plot show CFSE dilution in CD57⁺ or KLRG1⁺ CD8⁺ T cells gated on live CD3⁺ T cells in pp65 treated and untreated (medium only) groups. Bottom histograms show KLRG1 or CD57 expressions in corresponding three CD57 or KLRG1 subsets (numbered boxes) B: Summary of proliferation data from FIG. 2A. Proportion (%) of cells diluting CFSE (e.g., CFSE dilution indicated proliferation) for CD57⁺KLRG1⁻, CD57⁺KLRG1⁺, CD57⁻KLRG1⁺, and CD57⁻KLRG1⁻ subsets in the CMV pp65-specific CD8⁺ T cells (mean±SEM, two-tailed Wilcoxon-Mann-Withney used to generate p values). C: Proportions (%) of CMVpp65-specific CD8⁺ T cells, and CD57⁺KLRG1⁻, CD57⁺KLRG1⁺, CD57⁻KLRG1⁺ and CD57⁻KLRG1⁻ subsets (same as in FIG. 2B) from patients (n=3) in acute, contraction, and chronic phases of CMV infection. Samples for the contraction phase were collected approximately a month post-acute phase, and the chronic phase were collected at least a year after collection of the acute phase samples. D: Proliferation in vitro of CD57⁺ and KLRG1⁺ CD8⁺ T cells over 7 days in the presence or absence of CMV pp65 single peptide stimulation. Plots show CFSE dilution following proliferation of stimulated CMVpp65-specific CD8⁺ T cells, whereas no CFSE dilution by unstimulated control CMVpp65-specific CD8⁺ T cells. F: Preserved CD57 expression in a CFSE dilution assay when gated on total CMVpp65-specific CD8⁺ T cells indicates few proliferating cells in the CD57⁺ population. G: Right panel shows distribution (mean±SEM, n=10) of percent CD57⁺ and (mean±SEM, n=3) KLRG1⁺ CFSE diluting (e.g., proliferating) cells.

FIG. 3A-3F. CMV pp65-specific CD8⁺ effector responses during acute primary CMV infection. A-C: Pooled data from an LTR cohort (n=21) showing the correlation of pp65-specific CD8⁺ effector function (IFNγ, TNFα, and CD107a) and KLRG1⁺ (A) or T-BET⁺ (B) or CD57⁺ (C) during primary CMV infection. Correlation coefficients (“R”) and p-values were calculated using Spearman rank correlation test. D: Individual pie charts of PBMC showing T cell effector multifunction responses (IFNγ, TNFα, and CD107a) for CD8⁺KLRG1⁺ (left pie) and CD8⁺KLRG1⁻ (right pie) during acute primary CMV infection. E: Individual pie charts of PBMC showing T cell effector multifunction responses for CD8⁺ T-BET⁺ (left pie) and CD8⁺ T-BET⁻ (right pie). F: Individual pie charts of PBMC showing T cell effector multifunction responses for CD8⁺CD57⁺ (left pie) and CD8⁺CD57⁻ (right pie). D-F: Boolean analysis of the percentage of total and individual effector multifunctional subset responses for CD8⁺ T cells from KLRG1⁺ (gray bars) and KLRG1⁻ (black bars) or T-BET⁺ (gray bars) and T-BET⁻ (black bars) or CD57⁺ (gray bars) and CD57⁻ (black bars) (* denotes p<0.05).

FIG. 4A-4F. T-BET expression correlates with KLRG1 expression on CD8⁺ T cells in primary CMV infection. A: Expression profiles gated on live CD3⁺CD8⁺ T cells show co-expression of T-BET with KLRG1 or CD57 (n=21 individuals). B: Correlation between T-BET⁺ and KLRG1⁺ or T-BET⁺ and CD57⁺ in CD8⁺ T cells shows statistically significant correlation between T-BET⁺ and KLRG1⁺ CD8⁺ T cells. The black lines indicate regression lines. Correlation coefficients (“R”) and p-values were generated using Spearman's Rank Correlation test. C: Predicted T-BET binding sites with corresponding TFBS scores in KLRG1 promoter. D: Predicted T-BET binding sites with corresponding TFBS scores in CD57 promoter. E: 1272 base pairs (bp) of KLRG1 promoter with predicted T-BET binding sites in boxes. F: T-BET ChIP at various genomic loci including KLRG1 promoter, IFNγ (positive control; known T-BET binding), CD57 (B3GAT1), and GAPDH (negative control). T-BET binding was found at promoter sequences of T-BET targets KLRG1 and IFN-γ (positive control), while no T-BET binding was found at promoter sequences for CD57 (B3GAT1), IL-4 (negative control), or GAPDH (negative control) genes.

FIG. 5A-5F. KLRG1⁺ total CD8⁺ T cells and CMV-specific CD8⁺ T cells in patients with controlled CMV infection (“controllers”) and patients experiencing relapse of CMV infection (“relapsers”). A: Distribution of % KLRG1⁺ and additional six phenotypic markers expressed on total CD8⁺ T cells for controllers (circles) (n=7) and relapsers (squares) (n=8) (mean±SEM). Percentage of CD8⁺ T cells expressing KLRG1 was significantly different (p<0.002) between controllers and relapsers. B: Controller patients had significant enrichment for KLRG1⁺, T-BET⁺, and CD57⁺CD8⁺ T cells compared to relapser patients (controllers (n=12), and relapsers (n=11)). C: Percentage of T-BET⁺CD8⁺ T cells producing T cell effector molecules following stimulation revealed significant difference between controllers and relapsers. D: Percentage of KLRG1⁺ CD8⁺ T cells in producing T cell effector molecules following stimulation revealed significant difference between controllers and relapsers. E: Expression profiles of Dextramer⁺ (CMV pp65 stimulation) versus KLRG1⁺ revealed enrichment for KLRG1⁺ Dextramer⁺ T cells in controllers. The boxes represent KLRG1⁺ Dextramer⁺ C8⁺ T cells. F: Distribution of KLRG1⁺ and additional six phenotypic markers as expressed on total Dextramer⁺CD8⁺ T cells for controllers (circles) (n=7) and relapsers (squares) (n=8) (mean±SEM). Percentage of Dextramer⁺CD8⁺ T cells expressing KLRG1 was significantly different (p<0.003, Mann-Whitney-Wilcoxon) between controllers and relapsers.

FIG. 6A-6B. Total and CMV-specific CD8⁺ T cells in BAL samples express low levels of KLRG1 and T-BET. A: Phenotypic analysis of CMV-specific CD8⁺ T cells in BAL (upper panel) and Blood (lower panel) during acute/primary infection. Data is a representative example from one of six individuals. B: Summary of the phenotypic analysis. CMV-specific CD8⁺ T cells expressing KLRG1 and T-BET were reduced in BAL (solid bar) compared to PBMC (cross hatched bar), whereas CMV-specific CD8⁺ T cells expressing CD57⁺ were similar between the two sample types.

FIG. 7A-7F. A-B: Gating strategy for live CMV pp65-specific MHC Class I Dextramer⁺CD3⁺CD8⁺ cells and the phenotypic markers expressed in acute primary and chronic CMV infection. C-F: During primary infection, both total and CMV-specific T_{AEFF} expressed a CD27^{hi} CD28^{+/-}CD38⁺CD279⁺CD45RA⁻ acute T effector phenotype and in the transition to chronic CMV infection these cells progressed to a CD27^{lo/-}CD28⁻CD38⁻CD279⁻CD45RA^{+/hi} T effector memory (T_{EM}) phenotype.

### DETAILED DESCRIPTION

This document provides methods and materials for assessing mammals (e.g., humans) having a CMV infection. For example, this document provides methods and materials for assessing a mammal having a CMV infection (e.g., a CMV seronegative human who received CMV seropositive donor transplant tissue) to determine if the mammal contains a highly elevated level of KLRG1⁺/CD8⁺ T cells, thereby identifying that mammal as being highly likely to control the CMV infection, to determine if the mammal contains a moderately elevated level of KLRG1⁺/CD8⁺ T cells, thereby identifying that mammal as being likely to control the CMV infection, or to determine if the mammal lacks an elevated level of KLRG1⁺7 CD8⁺ T cells, thereby identifying that mammal as being unlikely to control the CMV infection.

As used herein, KLRG1 refers to a killer cell lectin-like subfamily G member 1. When assessing a human having a CMV infection to determine if the human contains an elevated level (e.g., a highly elevated level or a moderately elevated level) of KLRG1⁺/CD8⁺ T cells, the KLRG1 polypeptide can be a human KLRG1 polypeptide. Examples of human KLRG1 polypeptides include, without limitation, those polypeptides with the amino acid sequence set for in GenBank accession number NP_001316028.1. As used herein, CD3 refers to cluster of differentiation 3 polypeptide. When assessing a human having a CMV infection to determine if the human contains an elevated level (e.g., a highly elevated level or a moderately elevated level) of KLRG1⁺/CD8⁺ T cells, the CD3 can be a human CD3. Examples of a human CD3 polypeptides include, without limitation, those polypeptides with the amino acid sequence set for in GenBank accession number NP_000724.1. As used herein, CD8 refers to cluster of differentiation 8 polypeptide. When assessing a human having a CMV infection to determine if the human contains an elevated level (e.g., a highly elevated level or a moderately elevated level) of KLRG1⁺7 CD8⁺ T cells, the CD8 can be a human CD8. Examples of a human CD8 polypeptides include, without limitation, those polypeptides with the amino acid sequence set for in GenBank accession number NP_001139345.1.

Any appropriate mammal having a CMV infection can be assessed for the presence or absence of an elevated level (e.g., a highly elevated level or a moderately elevated level) of KLRG1⁺/CD8⁺ T cells as described herein. For example, humans and other primates such as monkeys having a CMV infection can be identified as having the presence or absence of an elevated level (e.g., a highly elevated level or a moderately elevated level) of KLRG1⁺/CD8⁺ T cells. In some cases, a CMV seronegative mammal (e.g., a human) having received transplant tissue (e.g., a solid organ transplant such as a lung, liver, heart, and/or kidney transplant) from a CMV seropositive donor can be identified as having the presence or absence of an elevated level (e.g., a highly elevated level or a moderately elevated level) of KLRG1⁺/CD8⁺ T cells as described herein. In some cases, a CMV seropositive mammal having received transplant tissue (e.g., a solid organ transplant such as a lung, liver, heart, and/or kidney transplant) from a CMV seropositive donor or a CMV seronegative donor can be identified as having the presence or absence of an elevated level (e.g., a highly elevated level or a moderately elevated level) of KLRG1⁺/CD8⁺ T cells as described herein.

When assessing a mammal (e.g., a human) having a CMV infection for the presence or absence of an elevated level (e.g., a highly elevated level or a moderately elevated level) of KLRG1⁺/CD8⁺ T cells, the mammal can have any type of CMV infection. For example, a mammal having a pre-acute CMV infection, an acute CMV infection, a CMV infection in contraction phase, or a chromic CMV infection can be identified as having the presence or absence of an elevated level (e.g., a highly elevated level or a moderately elevated level) of KLRG1⁺/CD8⁺ T cells as described herein. In some cases, a mammal suspected of having a CMV infection can be identified as having the presence or absence of an elevated level (e.g., a highly elevated level or a moderately elevated level) of KLRG1⁺/CD8⁺ T cells as described herein.

Any appropriate sample containing CD8⁺ T cells can be obtained from a mammal (e.g., a human) to identify that mammal as having the presence or absence of an elevated level (e.g., a highly elevated level or a moderately elevated level) of KLRG1⁺/CD8⁺ T cells. For example, blood samples can be obtained from a mammal and assessed to identify that mammal as having the presence or absence of an elevated level (e.g., a highly elevated level or a moderately elevated level) of KLRG1⁺/CD8⁺ T cells. In some cases, T cells (e.g., CD8⁺ T cells) can be extracted from a sample (e.g., a blood sample) obtained from a mammal to be assessed, and that extracted sample can be assessed to determine if the mammal has a presence or absence of an elevated level (e.g., a highly elevated level or a moderately elevated level) of KLRG1⁺/CD8⁺ T cells. Any appropriate method can be used to extract T cells (e.g., CD8⁺ T cells) from a sample (e.g., a blood sample such as a blood sample containing peripheral blood mononuclear cells (PBMCs)). For example, centrifugation using a density gradient, magnetic cell sorting, and/or fluorescent-activated cell sorting techniques can be performed to obtain a sample containing T cells (e.g., CD8⁺ T cells).

In some cases, a sample (e.g., a sample containing T cells) can be obtained from a mammal (e.g., a human) at any appropriate time and/or stage of a CMV infection. For example, a sample can be obtained from a mammal to be assessed as described herein prior to detection of a CMV infection (e.g., a pre-CMV infection or a pre-acute CMV infection). In cases where a mammal (e.g., a human) received transplant tissue (e.g., a solid organ transplant such as a lung, liver, heart, and/or kidney transplant), a sample can be obtained before, contemporaneously with, and/or after transplantation. In some cases, a sample can be obtained from a mammal (e.g., a human) having a pre-CMV infection prior to discontinuing an initial three-month antiviral prophylaxis treatment that may follow a transplantation procedure (e.g., a solid organ transplantation such as a lung transplantation). For example, a sample can be obtained from a mammal (e.g., a human) having received a lung transplant prior to the completion of an antiviral prophylaxis treatment. In another example, a sample can be obtained from a mammal (e.g., a human) prior to detection of a CMV infection independent of whether the mammal has received or will be receiving transplant tissue (e.g., a solid organ transplantation). In some cases, a sample to be assessed as described herein can be obtained from a mammal after detection of a CMV infection.

Once a sample containing T cells (e.g., CD8⁺ T cells) is obtained, that sample can be assessed to determine if the mammal contains the presence or absence of an elevated level (e.g., a highly elevated level or a moderately elevated level) of KLRG1⁺/CD8⁺ T cells. In some cases, total CD8⁺ T cells within a sample can be assessed to determine if the mammal contains an elevated level (e.g., a highly elevated level or a moderately elevated level) of KLRG1⁺/CD8⁺ T cells. For example, the percentage of CD8⁺ T cells that are KLRG1⁺/CD8⁺ T cells within a sample can be determined to identify the presence or absence of an elevated level (e.g., a highly elevated level or a moderately elevated level) of KLRG1⁺/CD8⁺ T cells. In some cases, a sub-population of CD8⁺ T cells within a sample can be assessed to determine if the mammal contains an elevated level (e.g., a highly elevated level or a moderately elevated level) of KLRG1⁺/CD8⁺ T cells. For example, the percentage of CMV-specific CD8⁺ T cells that are KLRG1⁺/CD8⁺ T cells within a sample can be determined to identify the presence or absence of an elevated level (e.g., a highly elevated level or a moderately elevated level) of KLRG1⁺/CD8⁺ T cells.

Any appropriate method can be used to determine the number or percentage of particular cells (e.g., CD8⁺ T cells, KLRG1⁺/CD8⁺ T cells, KLRG1⁻/CD8⁺ T cells, CMV-specific CD8⁺ T cells, CMV-specific KLRG1⁺/CD8⁺ T cells, and/or CMV-specific KLRG1⁻/CD8⁺ T cells) within a sample (e.g., a blood sample). For example, flow cytometry can be used to determine the number or percentage of particular cells (e.g., CD8⁺ T cells, KLRG1⁺/CD8⁺ T cells, KLRG1⁻/CD8⁺ T cells, CMV-specific CD8⁺ T cells, CMV-specific KLRG1⁺/CD8⁺ T cells, and/or CMV-specific KLRG1⁻/CD8⁺ T cells) within a sample (e.g., a blood sample). Any appropriate flow cytometric method can be used to analyze cells. In some cases, fluorescent agents such as carboxyfluorescein succinimidyl ester (CFSE), fluorescently-labeled antibodies (e.g., fluorescently-labeled anti-CD3 antibodies, fluorescently-labeled anti-CD4 antibodies, fluorescently-labeled anti-CD8 antibodies, fluorescently-labeled anti-CD27 antibodies, fluorescently-labeled anti-CD57 antibodies, fluorescently-labeled anti-CD44 antibodies, fluorescently-labeled anti-KLRG1 antibodies, and/or fluorescently-labeled anti-CD62L antibodies), fluorescently-labeled peptide-tetramer complexes (e.g., fluorescently-labeled viral antigen-tetramer complexes), or combinations thereof can be used to stain T cells for analysis. For example, fluorescently-labeled anti-CD3 antibodies (e.g., fluorescein-labeled anti-CD3 antibodies such as FITC-OKT3) and fluorescently-labeled anti-CD8 antibodies (e.g., phycoerythrin-labeled anti-CD8 antibodies such as PE-SK1) can be used to stain CD8⁺ T cells, and fluorescently-labeled anti-CD3 antibodies (e.g., fluorescein-labeled anti-CD3 antibodies such as FITC-OKT3), fluorescently-labeled anti-CD8 antibodies (e.g., phycoerythrin-labeled anti-CD8 antibodies such as PE-SK1), and fluorescently-labeled anti-KLRG1 antibodies (e.g., allophycocyanin-labeled anti-KLRG1 antibodies such as APC-KLRG1) can be used to stain KLRG1⁺/CD8⁺ T cells. In some cases, fluorescently-labeled anti-IFNγ, fluorescently-labeled anti-TNFα, and/or fluorescently-labeled anti-CD107a can be used to assess immune cell effector function. Examples of fluorescent labels that can be used during analysis include, without limitation, fluorescein, phycoerythrin, Cy3, Cy5, Rhodamine, Alexa 488, and Brilliant Violet. In some cases, one or more fluorescent agents (e.g., fluorescently-labeled antibodies) other than those designed and used to help identify T cells (e.g., CD8⁺ T cells) or particular types of T cells (e.g., CD4⁺) can be included during the analysis to provide additional information about the phenotype of the analyzed T cells even though the fluorescent signals from those fluorescent agents may or may not be used to identify the level of KLRG1⁺/CD8⁺ T cells (e.g., the percentage of CD8⁺ T cells that are KLRG1⁺/CD8⁺ T cells) within a sample. For example, fluorescent agents (e.g., fluorescently labeled antibodies) that bind to surface proteins or markers such as PD-1, TIM3, LAG3, CD28, CD152, CD44, CD69, CD107a, CD11b, CD62L, CD127, CD30, and/or CD45RA/CDR450 can be used to capture expression information about those surface proteins or markers by each analyzed T cell.

In some cases, fluorescent-activated cell sorting techniques using antibodies such as anti-CD3 antibodies, anti-CD8 antibodies, and anti-KLRG1 antibodies can be used to determine the percentage of CD8⁺ T cells that are KLRG1⁺/CD8⁺ T cells within a sample, while fluorescent-activated cell sorting techniques using antibodies such as anti-CD3 antibodies, anti-CD8 antibodies, and anti-KLRG1 antibodies and dextramers such as a CMV pp65-specific dextramer can be used to determine the percentage of CMV-specific CD8⁺ T cells that are KLRG1⁺/CD8⁺ T cells within a sample. In some cases, immunohistochemistry (IHC) techniques, ELISAs, immunofluorescence (IF) techniques, and/or Western blot techniques can be used to determine if a sample contains the presence or absence of an elevated level (e.g., a highly elevated level or a moderately elevated level) of KLRG1⁺/CD8⁺ T cells.

Examples of anti-CD3 antibodies that can be used as described herein to determine the number or percentage of T cells within a sample include, without limitation, anti-CD3 monoclonal antibodies from Invitrogen (Catalog No. 14-0037-82) and anti-CD3 antibodies from BD Biosciences (e.g., clone UCHT1). Examples of anti-CD8 antibodies that can be used as described herein to determine the number or percentage of CD8⁺ T cells within a sample include, without limitation, anti-CD8 polyclonal antibodies from Novus Biologicals (Catalog No. NBP2-29475) and anti-CD8 antibodies from BD Biosciences (e.g., clone SK1). Examples of anti-KLRG1 antibodies that can be used as described herein to determine the number or percentage of KLRG1⁺ cells (e.g., KLRG1⁺/CD8⁺ T cells) within a sample include, without limitation, anti-KLRG1 antibodies obtained from R&D Systems (Catalog No. MAB70293), anti-KLRG1 polyclonal antibodies obtained from Invitrogen (Catalog No. PAS-67424), and anti-KLRG1 antibodies obtained from EBiosciences (e.g., clone 13F12F2).

Any appropriate flow cytometer can be used to analyze the cells present in the sample. Examples of such analyzers include, without limitation, Attune NxT (Thermo Fisher), BD analyzers (e.g., Accuri, FACSCelesta, FACSymphony, FACSCanto II, LSRFortessa, LSRFortessa X-20, and FACSVerse), Quanteon (NovoCyte), Image StreamX (Luminex), MACSQuant (Miltenyl), Gallios (Beckman), and NucleoCounter NC-3000 (ChemoMetec).

Once the percentage of total CD8⁺ T cells that are KLRG1⁺/CD8⁺ T cells (or the percentage of CMV-specific CD8⁺ T cells that are KLRG1⁺/CD8⁺ T cells) is determined, that percentage can be compared to one or more cutoff percentages to determine whether or not the mammal has a highly elevated level of KLRG1⁻/CD8⁺ T cells, to determine whether or not the mammal has a moderately elevated level of KLRG1⁺/CD8⁺ T cells, or to determine whether or not the mammal lacks an elevated level of KLRG1⁺/CD8⁺ T cells. The term “highly elevated level” as used herein with respect to the level of KLRG1⁺/CD8⁺ T cells within a mammal refers to a percentage of CD8⁺ T cells (e.g., percentage of total CD8⁺ T cells or percentage of CMV-specific CD8⁺ T cells) within a sample obtained from that mammal that are KLRG1⁻/CD8⁺ T cells that is more than 50 percent (e.g., at least 51, 50, 55, 65, 75, 80, 90, or 100 percent). For example, determining that a sample obtained from a mammal (e.g., a mammal having a CMV infection) contains KLRG1⁺/CD8⁺ T cells that make up more than 50 percent (e.g., at least 51, 50, 55, 65, 75, 80, 90, or 99 percent) of the total CD8⁺ T cell population of that sample can indicate that that mammal contains a highly elevated level of KLRG1⁺/CD8⁺ T cells. The term “moderately elevated level” as used herein with respect to the level of KLRG1⁺/CD8⁺ T cells within a mammal refers to a percentage of CD8⁺ T cells (e.g., percentage of total CD8⁺ T cells or percentage of CMV-specific CD8⁺ T cells) within a sample obtained from that mammal that are KLRG1⁺/CD8⁺ T cells that is more than 40 percent and less than or equal to 50 percent (e.g., 41, 42, 43, 44, 45, 46, 47, 48, 49, or 50 percent). For example, determining that a sample obtained from a mammal (e.g., a mammal having a CMV infection) contains KLRG1⁺/CD8⁺ T cells that make up more than 40 percent and less than or equal to 50 percent (e.g., 41, 42, 43, 44, 45, 46, 47, 48, 49, or 50 percent) of the total CD8⁺ T cell population of that sample can indicate that that mammal contains a moderately elevated level of KLRG1⁻/CD8⁺ T cells. Determining that a sample obtained from a mammal (e.g., a mammal having a CMV infection) contains KLRG1⁺/CD8⁺ T cells that make up less than 40 percent (e.g., less than 40, 35, 25, 15, 10, or 5 percent) of the total CD8⁺ T cell population of that sample can indicate that that mammal lacks an elevated level of KLRG1⁺/CD8⁺ T cells. In another example, determining that a sample obtained from a mammal (e.g., a mammal having a CMV infection) contains CMV-specific KLRG1⁻/CD8⁺ T cells that make up more than 50 percent (e.g., at least 51, 55, 65, 75, 80, 90, or 99 percent) of the CMV-specific CD8⁺ T cell population of that sample can indicate that that mammal contains a highly elevated level of KLRG1⁺/CD8⁺ T cells. Determining that a sample obtained from a mammal (e.g., a mammal having a CMV infection) contains CMV-specific KLRG1⁺/CD8⁺ T cells that make up more than 40 percent and less than or equal to 50 percent (e.g., 41, 42, 43, 44, 45, 46, 47, 48, 49, or 50 percent) of the CMV-specific CD8⁺ T cell population of that sample can indicate that that mammal has a moderately elevated level of KLRG1⁺/CD8⁺ T cells. Determining that a sample obtained from a mammal (e.g., a mammal having a CMV infection) contains CMV-specific KLRG1⁺/CD8⁺ T cells that make up less than 40 percent (e.g., less than 40, 35, 25, 15, 10, or 5 percent) of the CMV-specific CD8⁺ T cell population of that sample can indicate that that mammal lacks an elevated level of KLRG1⁺/CD8⁺ T cells.

Once a mammal (e.g., a human having a CMV infection) is identified as having the presence of a highly elevated level of KLRG1⁻/CD8⁺ T cells as described herein, that mammal can be classified as being highly likely to control a CMV infection. For example, a human (e.g., a CMV seronegative human who received CMV seropositive donor transplant tissue) that is identified as having a highly elevated level of KLRG1⁺/CD8⁺ T cells as described herein can be classified as being highly likely to control a CMV infection.

Once a mammal (e.g., a human having a CMV infection) is identified as having the presence of a moderately elevated level of KLRG1⁺/CD8⁺ T cells as described herein, that mammal can be classified as being likely to control a CMV infection. For example, a human (e.g., a CMV seronegative human who received CMV seropositive donor transplant tissue) that is identified as having a moderately elevated level of KLRG1⁺/CD8⁺ T cells as described herein can be classified as being likely to control a CMV infection.

Once a mammal (e.g., a human having a CMV infection) is identified as having the absence of an elevated level of KLRG1⁺/CD8⁺ T cells as described herein, that mammal can be classified as being unlikely to control a CMV infection. For example, a human (e.g., a CMV seronegative human who received CMV seropositive donor transplant tissue) that is identified as lacking an elevated level of KLRG1⁺/CD8⁺ T cells as described herein can be classified as being unlikely to control a CMV infection.

This document also provides methods and materials for treating a mammal having a CMV infection (or suspected of having a CMV infection). For example, a mammal (e.g., a human having a CMV infection) that was identified as lacking an elevated level of KLRG1⁺/CD8⁺ T cells as described herein can be treated with one or more antiviral agents. In some cases, a mammal (e.g., a human having a CMV infection) that was identified as lacking an elevated level of a KLRG1⁺/CD8⁺ T cells as described herein can be administered, or instructed to self-administer, one or more antiviral agents to control the viral infection.

Any appropriate antiviral agents can be administered to a mammal (e.g., a human having a CMV infection who was identified as lacking an elevated level of KLRG1⁺/CD8⁺ T cells) to treat a CMV infection. In some cases, an antiviral agent used as described herein to treat a CMV infection can reduce one or more symptoms of CMV infection within a mammal (e.g., viremia, fatigue, cough, diarrhea, or combinations thereof). Examples of antiviral agents that can be used as described herein to treat a CMV infection in a mammal (e.g., a human) that was identified as lacking an elevated level of KLRG1⁺/CD8⁺ T cells include, without limitation, ganciclovir, valganciclovir, foscarnet, cidofovir, and letermovir.

In some cases, one or more (e.g., one, two, three, four, five, or more) antiviral agents can be formulated into a pharmaceutically acceptable composition for administration to a mammal (e.g., a human) that has a CMV infection and was identified as lacking an elevated level of KLRG1⁺/CD8⁺ T cells to reduce one or more symptoms of the CMV infection (e.g., viremia, fatigue, cough, diarrhea, or combinations thereof) within that mammal. For example, a therapeutically effective amount of an antiviral agent can be formulated together with one or more pharmaceutically acceptable carriers (additives) and/or diluents. A pharmaceutical composition can be formulated for administration in solid or liquid form including, without limitation, in the form of sterile solutions, suspensions, sustained-release formulations, tablets, capsules, pills, powders, or granules.

Pharmaceutically acceptable carriers, fillers, and vehicles that can be used in a pharmaceutical composition containing one or more antiviral agents as described herein can include, without limitation, ion exchangers, alumina, aluminum stearate, lecithin, serum proteins (e.g., human serum albumin), buffer substances (e.g., phosphates), glycine, sorbic acid, potassium sorbate, partial glyceride mixtures of saturated vegetable fatty acids, water, salts or electrolytes (e.g., protamine sulfate), disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

A pharmaceutical composition containing one or more antiviral agents can be designed for oral or parenteral (including subcutaneous, intramuscular, intravenous, and/or intradermal) administration. When being administered orally, a pharmaceutical composition can be in the form of a pill, tablet, or capsule. Compositions suitable for parenteral administration can include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and can be stored in a freeze dried (lyophilized) condition requiring the addition of the sterile liquid carrier, for example, water for injections, immediately prior to use. Extemporaneous injection solutions and suspensions can be prepared from sterile powders, granules, and tablets.

In some cases, a pharmaceutically acceptable composition including one or more antiviral agents can be administered locally or systemically. For example, a composition provided herein can be administered locally by intravenous injection or blood infusion. In some cases, a composition provided herein can be administered systemically, orally, or by injection to a mammal (e.g., a human).

In some cases, an effective amount of a composition containing one or more antiviral agents described herein can be any amount that reduces one or more symptoms of a CMV infection (e.g., viremia, fatigue, cough, diarrhea, or combinations thereof) within a mammal (e.g., a human having a CMV infection) that was identified as lacking an elevated level of KLRG1⁺/CD8⁺ T cells without producing significant toxicity to the mammal. In some cases, treatment of a mammal (e.g., a human) infected with CMV and identified as lacking an elevated level of KLRG1⁺/CD8⁺ T cells can include administering an effective amount of oral valganciclovir and/or intravenous ganciclovir. For example, a human infected with CMV and identified as lacking an elevated level of KLRG1⁺/CD8⁺ T cells can be administered an effective amount of oral valganciclovir. In another example, a human infected with CMV and identified as lacking an elevated level of KLRG1⁺/CD8⁺ T cells can be administered an effective amount of intravenous ganciclovir.

In some cases, treatment of a mammal (e.g., a human having a CMV infection) that was identified as lacking an elevated level of KLRG1⁺/CD8⁺ T cells can include administering an antiviral agent (e.g., oral valganciclovir) in an amount from about 1 mg to about 20 mg per kg of body weight per day. In some cases, an effective amount of valganciclovir to treat a CMV infection in a mammal identified as lacking an elevated level of KLRG1⁺/CD8⁺ T cells can be from about 250 mg to about 1.75 g (e.g., from about 250 mg to about 1.5 g, from about 250 mg to about 1.25 g, from about 250 mg to about 1.0 g, from about 250 mg to about 750 mg, from about 500 mg to about 1.75 mg, from about 500 mg to about 1.5 mg, from about 500 mg to about 1.25 mg, from about 500 mg to about 1.0 mg, from about 500 mg to about 750 mg, from about 750 mg to about 1.75 mg, from about 750 mg to about 1.5 g, from about 1 g to about 1.75 g, from about 1 g to about 1.5 g, or from about 1.25 g to about 1.5 g) administered orally per day.

In some cases, treatment of a mammal (e.g., a human having a CMV infection) that was identified as lacking an elevated level of KLRG1⁺/CD8⁺ T cells can include administering intravenous ganciclovir in an amount from about 100 μg to about 20 mg per kg of body weight per day. In some cases, an effective amount of ganciclovir to treat CMV infection in a mammal identified as lacking an elevated level of KLRG1⁺/CD8⁺ T cells can be from about 2.5 mg/kg to about 10 mg/kg (e.g., from about 2.5 mg/kg to about 9 mg/kg, from about 2.5 mg/kg to about 8 mg/kg, from about 2.5 mg/kg to about 7 mg/kg, from about 2.5 mg/kg to about 6 mg/kg, from about 2.5 mg/kg to about 5 mg/kg, from about 2.5 mg/kg to about 4 mg/kg, from about 2.5 mg/m² to about 4 mg/kg, or from about 2.5 mg/kg to about 3 mg/kg) administered intravenously per day.

In some cases, if a particular mammal infected with CMV and identified as lacking an elevated level of KLRG1⁺/CD8⁺ T cells fails to respond to a particular amount, then the amount of an antiviral agent can be increased by, for example, two fold. After receiving this higher amount, the mammal can be monitored for both responsiveness to the treatment and toxicity symptoms, and adjustments made accordingly. In some cases, the effective amount of a composition containing one or more antiviral agents can remain constant or can be adjusted as a sliding scale or variable dose depending on the mammal's response to treatment. Various factors can influence the actual effective amount used for a particular application. For example, the use (or lack thereof) of an immunosuppressant treatment regimen, the frequency of administration, duration of treatment, use of multiple treatment agents, route of administration, tolerance to the medication, and severity of the condition can require an increase or decrease in the actual effective amount administered.

A frequency of administration of a composition containing one or more antiviral agents as described herein can be any frequency that reduces one or more symptoms of a CMV infection (e.g., viremia, fatigue, cough, diarrhea, or combinations thereof) within a mammal (e.g., a human) having a CMV infection and identified as lacking an elevated level of KLRG1⁺/CD8⁺ T cells without producing significant toxicity to the mammal. In some cases, a mammal (e.g., a human having a CMV infection) that was identified as lacking an elevated level of KLRG1⁺/CD8⁺ T cells can be administered an antiviral agent (e.g., oral valganciclovir) at a frequency ranging from about two times a day to about 12 doses a month. For example, treatment of a mammal (e.g., a human having a CMV infection) that was identified as lacking an elevated level of KLRG1⁺/CD8⁺ T cells can include administering an antiviral agent (e.g., valganciclovir) orally twice a day. In some cases, the frequency of administration of an antiviral agent (e.g., valganciclovir) can be twice a day, once a day, twice every other day, once every other day, or three times a week. The frequency of administration of an antiviral agent (e.g., valganciclovir) can remain constant or can be variable during the duration of treatment. In some cases, treatment of a mammal (e.g., a human having a CMV infection) that was identified as lacking an elevated level of KLRG1⁺/CD8⁺ T cells can include administration of ganciclovir every 12 hours. In some cases, the frequency of administration of an antiviral agent (e.g., ganciclovir) can once every 24 to 48 hours. As with the effective amount, various factors can influence the frequency of administration used for a particular application. For example, the use (or lack thereof) of an immunosuppressant treatment regimen, the effective amount, duration of treatment, use of multiple treatment agents, route of administration, renal and/or bone marrow function, tolerance to the medication, and severity of the condition may require an increase or decrease in administration frequency.

An effective duration for administering a composition containing one or more antiviral agents can be any duration that reduces one or more symptoms of the CMV infection (e.g., viremia, fatigue, cough, diarrhea, or combinations thereof) within a mammal having a CMV infection and identified as lacking an elevated level of KLRG1⁺/CD8⁺ T cells without producing significant toxicity to the mammal. In some cases wherein a mammal (e.g., a human) receives a transplant (e.g., a lung transplant), the mammal can be treated by administering one or more antiviral agents for the initial three to 12 months following the transplantation (e.g., a prophylactic antiviral treatment using ganciclovir and/or valganciclovir). In some cases, one or more antiviral agents can be administered until two consecutive CMV measurements (e.g., CMV qPCR) as described herein are below an acceptable threshold of copies of CMV (e.g., less than 300 copies/mL). For example, treatment of a mammal (e.g., a human having a CMV infection) that was identified as lacking an elevated level of KLRG1⁺/CD8⁺ T cells can include administration of oral valganciclovir or intravenous ganciclovir until two consecutive (e.g., weekly) CMV measurements are below an acceptable threshold (e.g., less than 300 copies of CMV/mL).

In some cases, a mammal (e.g., a human having a CMV infection) that was identified as lacking an elevated level of KLRG1⁺/CD8⁺ T cells can be administered one or more antiviral agents for any appropriate duration that reduces one or more symptoms of the CMV infection (e.g., viremia, fatigue, cough, diarrhea, or combinations thereof) or maintains control over the CMV infection. In some cases, the effective duration can vary from about six weeks to about three months or can be for as long as the mammal is alive. In some cases, the effective duration can extend until the mammal has two consecutive (e.g., weekly) CMV measurements that are below an acceptable threshold (e.g., less than 300 copies of CMV/mL). Multiple factors can influence the effective duration used for a particular treatment. For example, an effective duration can vary with the frequency of administration, the use (or lack thereof) of an immunosuppressant treatment regimen, effective amount, use of multiple treatment agents, route of administration, tolerance to the medication, and severity of the condition being treated.

In some cases, a treatment for a mammal (e.g., a human) infected with CMV (e.g., a CMV seronegative human who received CMV seropositive donor transplant tissue) that was identified as lacking an elevated level of KLRG1⁺/CD8⁺ T cells can be as set forth in Table 1.

TABLE 1
Exemplary CMV treatment options for mammals
(e.g., humans) identified as lacking an elevated level of
KLRG1⁺/CD8⁺ T cells.

| Treatment | Antiviral | |
|---|---|---|
| Option # | Agent | Dosage |
| 1 | Valganciclovir | From about 100 mg to about 600 mg (e.g., |
| | | 450 mg) once to twice a day |
| 2 | Valganciclovir | From about 450 mg to about 900 mg once a |
| | | day |
| 3 | Ganciclovir | From about 2.5 mg/kg to about 5 mg/kg |
| | | daily (adjusted for renal function) |
| 4 | Foscarnet | From about 40 mg/kg to about 120 mg/kg |
| | | daily (adjusted for renal function) |
| 5 | Foscarnet | From about 50 mg/kg to about 100 mg/kg |
| | | daily (adjusted for renal function) |

In some cases, a mammal (e.g., a human) infected with CMV (e.g., a CMV seronegative human who received CMV seropositive donor transplant tissue) and identified as lacking an elevated level of KLRG1⁺/CD8⁺ T cells can be administered one or more antiviral agents and one or more immunosuppressants. Examples of immunosuppressants that can be used in combination with one or more antiviral agents as described herein include, without limitation, tacrolimus, prednisone, everolimus, mycophenolate mofetil (e.g., CellCept®), and azathioprine. In some cases, a human infected with CMV (e.g., a CMV seronegative human who received CMV seropositive donor transplant tissue) and identified as lacking an elevated level of KLRG1⁺/CD8⁺ T cells can be treated as set forth in Table 2.

TABLE 2
Treatment options using a combinations of one or more
immunosuppressants and one or more antiviral agents.

| Combination | | | Treatment |
|---|---|---|---|
| Treatment | | | Option # |
| Option # | Immunosuppressant | Dosage | from Table 1 |
| 1 | Tacrolimus | From about 1 mg to about 10 mg (e.g., | 1 |
| | | 5 mg) twice daily (e.g., about once | 2 |
| | | every 12 hours) for about 2 to 4 | 3 |
| | | months (e.g., 3 months) | 4 |
| | | | 5 |
| 2 | Prednisone | From about 5 mg to about 40 mg per | 1 |
| | | day for about 2 to 4 months (e.g., 3 | 2 |
| | | months) | 3 |
| | | | 4 |
| | | | 5 |
| 3 | Mycophenolate | From about 250 mg to about 1500 | 1 |
| | mofetil | mg two times a day for about 2 to | 2 |
| | | 4 months (e.g., 3 months) | 3 |
| | | | 4 |
| | | | 5 |
| 4 | Azathioprine | From about 50 mg to about 150 mg | 1 |
| | | once a day for about 2 to 4 months | 2 |
| | | (e.g., 3 months) | 3 |
| | | | 4 |
| | | | 5 |
| 5 | Everolimus | From about 0.75 mg to about 4 mg | 1 |
| | | two times a day for about 2 to 4 | 2 |
| | | months (e.g., 3 months) | 3 |
| | | | 4 |
| | | | 5 |

In some cases, one or more antiviral agents and one or more immunosuppressants can be administered to a mammal (e.g., a human) before, contemporaneously with, or immediately after receiving transplant tissue. As described herein, mammals (e.g., humans) infected with CMV (e.g., a CMV seronegative human received CMV seropositive donor transplant tissue) and identified as lacking an elevated level of KLRG1⁺/CD8⁺ T cells can be treated by administering oral valganciclovir and/or intravenous ganciclovir and an immunosuppressant such as tacrolimus.

As described herein, mammals (e.g., humans) infected with CMV (e.g., a CMV seronegative human received CMV seropositive donor transplant tissue) that were identified as having a moderately elevated level or a highly elevated level of KLRG1⁺/CD8⁺ T cells can be treated in a manner that avoids the administration of antiviral agents while being monitored for the emergence of an uncontrolled CMV infection. If one or more signs of an uncontrolled CMV infection emerge while the mammal identified as having a moderately elevated level or a highly elevated level of KLRG1⁺/CD8⁺ T cells is not being administered an antiviral agent, then that mammal can be administered one or more antiviral agents as described herein to control the CMV infection. For example, when a mammal (e.g., a human) infected with CMV (e.g., a CMV seronegative human received CMV seropositive donor transplant tissue) and identified as having a moderately elevated level or a highly elevated level of KLRG1⁺/CD8⁺ T cells is detected as having an uncontrolled (e.g., active) CMV infection, the mammal can be administered one or more antiviral agents as described herein for mammals identified as lacking an elevated level of KLRG1⁺/CD8⁺ T cells (see, e.g., Table 1).

A mammal (e.g., a human) infected with CMV (e.g., a CMV seronegative human received CMV seropositive donor transplant tissue) that was identified as having a moderately elevated level or a highly elevated level of KLRG1⁺/CD8⁺ T cells can be monitored at any appropriate time to detect the emergence of an uncontrolled CMV infection. For example, a mammal (e.g., a human) infected with CMV (e.g., a CMV seronegative human received CMV seropositive donor transplant tissue) that was identified as having a moderately elevated level or a highly elevated level of KLRG1⁺/CD8⁺ T cells can be monitored about once every 4 to 12 weeks (e.g., about once every month) to determine whether or not the mammal has an uncontrolled CMV infection. In some cases, a mammal (e.g., a CMV seronegative human who received CMV seropositive donor transplant tissue) identified as having a moderately elevated level or a highly elevated level of KLRG1⁺/CD8⁺ T cells can be monitored at least once every one to three months while being administered one or more immunosuppressants and then less frequently (e.g., once every three to six months) when no longer being administered any immunosuppressants.

In some cases, a mammal (e.g., a CMV seronegative human who received CMV seropositive donor transplant tissue) identified as having a moderately elevated level of KLRG1⁺/CD8⁺ T cells can be monitored more frequently than the monitoring frequency used to monitor a mammal (e.g., a CMV seronegative human who received CMV seropositive donor transplant tissue) identified as having a highly elevated level of KLRG1⁺/CD8⁺ T cells. For example, a mammal (e.g., a CMV seronegative human who received CMV seropositive donor transplant tissue) identified as having a moderately elevated level of KLRG1⁺/CD8⁺ T cells can be monitored about once a month, while a mammal (e.g., a CMV seronegative human who received CMV seropositive donor transplant tissue) identified as having a highly elevated level of KLRG1⁺/CD8⁺ T cells can be monitored about once every three months.

Signs of an uncontrolled CMV infection include, without limitation, the emergence of recurrent episodes of viremia, end organ disease (e.g., colitis), antiviral resistance, and combinations thereof.

In some cases, a mammal (e.g., a human having a CMV infection) that was identified as having a moderately elevated level or a highly elevated level of KLRG1⁺/CD8⁺ T cells can be monitored instead of being administered an antiviral agent. In some cases, while monitoring a mammal (e.g., a human having a CMV infection) that was identified as having a moderately elevated level or a highly elevated level of KLRG1⁺/CD8⁺ T cells, clinical symptoms (e.g., fever, leukopenia, viremia, or combinations thereof) can reappear and/or an increase in CMV copy number can be detected (e.g., greater than 300 copies of CMV/mL). In such cases, the mammal (e.g., human) can be switched from a monitoring approach to a treatment approach that includes the administration of one or more antiviral agents as described herein. In some cases, a sample can be obtained from a mammal (e.g., a human having a CMV infection) in order to re-assess whether or not the mammal has a moderately elevated level or a highly elevated level of KLRG1⁺/CD8⁺ T cells.

As described herein, a course of treatment and/or the severity of one or more symptoms related to a CMV infection can be monitored for mammals (e.g., humans) whether the mammal has or lacks an elevated level of KLRG1⁺/CD8⁺ T cells. Any appropriate method can be used to determine whether a CMV infection is being treated or maintained in check. For example, samples can be collected from a mammal (e.g., a human having a CMV infection) at any appropriate time (e.g., daily, weekly, or monthly) and assessed.

In some cases, CMV infections can be monitored using quantitative PCR (qPCR or CMV qPCR). For example, a CMV infection can be monitored by performing qPCR on DNA extracted from blood of a mammal (e.g., a human) having a CMV infection. In some cases where qPCR is used to monitor a CMV infection, greater than 300 copies of CMV per mL of blood can be used as a threshold to identify an active CMV infection. In some cases, commercially available kits can be used to assess CMV infections using qPCR. Examples of such kits include, without limitation, EraGen Multicode (Luminex), Focus Simplexa (Focus Diagnostics), Elitech MGB Alert (Fisher Scientific), LightCycler (LC) CMV UL54 (Roche Molecular), and Abbott CMV (Abbott). In some cases, CMV infections can be monitored using RT-PCR. For example, a CMV infection can be monitored by subjecting RNA extracted from the blood of a mammal (e.g., a human) having a CMV infection to reverse transcription (RT) followed by PCR amplification.

In some cases, CMV infections can be monitored by detecting the presence of CMV viral proteins in an appropriate sample (e.g., blood sample) from a mammal (e.g., a human) having a CMV infection. In some cases, viral proteins can be monitored using complement fixation, enzyme-linked immunosorbent assay (ELISA), anti-complement fluorescence, and indirect hemagglutination. In some cases, CMV viral proteins that can be used to monitor CMV infections include, without limitation, CMV-G, CMV Ext-2, CMV-M, pp28, pp38, pp52, pp65, pp72, and pp150. For example, a pp65 antigenemia assay can be used to detect and/or monitor a CMV infection using a blood sample of a mammal (e.g., a human) having a CMV infection. An example of a commercially available pp65 antigenemia kit that can be used as described herein includes, without limitation, the CINA Kit (ArgeneBiosoft).

In some cases, a mammal's (e.g., a human's) own antibodies present within the mammal's blood can be used to detect and/or monitor a CMV infection. For example, detection of a CMV-specific IgG using an avidity assay can be used to detect and/or monitor a CMV infection in a mammal. In some cases, a mammal can produce anti-CMV IgG antibodies after about 10-14 days of CMV infection. The levels anti-CMV IgG antibodies can rise during acute infection and then stabilize as the CMV infection is controlled either by an antiviral agent or by the mammal's immune system. In some cases, detection of anti-CMV IgM antibodies produced by a mammal's immune system can be used to detect and/or monitor a CMV infection. For example, detection of anti-CMV IgM antibodies using an ELISA can be used to detect and/or monitor a CMV infection in a mammal. Anti-CMV IgM antibodies can develop within about 1-2 weeks after initial CMV infection. In some cases, detecting and/or monitoring a CMV infection can include detecting a mammal's Type-1 antiviral immunity. Methods of monitoring a mammal's (e.g., a human's) Type-1 antiviral immunity can include, without limitation, monitoring production of immune effector molecules such as IFNγ, TNFα, and CD107a (via, e.g., intracellular cytokine staining of CD8⁺ T cells); monitoring major histocompatibility complex multi-mer staining; monitoring CD8⁺ T cell responses using a QuantiFERON-CMV assay; and monitoring CD4⁺ and CD8⁺ T cell responses using an enzyme-linked immunosorbent spot (ELISPOT) assay.

In some cases, a mammal (e.g., a human) infected with CMV (e.g., a CMV seronegative human received CMV seropositive donor transplant tissue) and identified as having a moderately elevated level or a highly elevated level of KLRG1⁺/CD8⁺ T cells that was successfully administered one or more antiviral agents as described herein to treat an uncontrolled CMV infection that emerged can be removed from antiviral therapy and monitored for possible re-emergence of an uncontrolled CMV infection. Each time an uncontrolled CMV infection emerges in a mammal (e.g., a human) infected with CMV (e.g., a CMV seronegative human received CMV seropositive donor transplant tissue) and identified as having a moderately elevated level or a highly elevated level of KLRG1⁺/CD8⁺ T cells that mammal can be treated with one or more antiviral agents as described herein (see, e.g., Table 1).

In some cases, a mammal (e.g., a human) infected with CMV (e.g., a CMV seronegative human received CMV seropositive donor transplant tissue) that was identified as having a moderately elevated level or a highly elevated level of KLRG1⁺/CD8⁺ T cells can be administered an antiviral agent in a less extensive manner than that used for a mammal that was identified as lacking an elevated level of KLRG1⁺/CD8⁺ T cells. For example, a mammal (e.g., a human) infected with CMV (e.g., a CMV seronegative human who received CMV seropositive donor transplant tissue) that was identified as having a moderately elevated level or a highly elevated level of KLRG1⁺/CD8⁺ T cells can be under the care of a physician in a manner that avoids the administration of any antiviral agents. In some cases, a human infected with CMV (e.g., a CMV seronegative human who received CMV seropositive donor transplant tissue) who was identified as having a moderately level or a highly elevated level of KLRG1⁺/CD8⁺ T cells can be monitored in a manner that avoids the administration of any antiviral agents for treatment of the CMV infection. In some cases, a human infected with CMV (e.g., a CMV seronegative human who received CMV seropositive donor transplant tissue) who was identified as having a moderately elevated level or a highly elevated level of KLRG1⁺/CD8⁺ T cells can be instructed to avoid the administration of any antiviral agents for treatment of the CMV infection. In some cases, a human infected with CMV (e.g., a CMV seronegative human who received CMV seropositive donor transplant tissue) who was identified as having a moderately elevated level or a highly elevated level of KLRG1⁺/CD8⁺ T cells can be administered one or more immunosuppressants and not administered any antiviral agents for treatment of the CMV infection.

This document also provides kits for determining the level of KLRG1⁺/CD8⁺ T cells present within a mammal (e.g., a human). For example, a kit provided herein can be used to determine if a mammal (e.g., a human infected with CMV) has the presence or absence of a moderately elevated level of KLRG1⁺/CD8⁺ T cells as described herein. In some cases, a kit provided herein can be used to determine if a mammal (e.g., a human infected with CMV) has the presence or absence of a highly elevated level of KLRG1⁺/CD8⁺ T cells as described herein.

A kit provided herein can include an antibody that specifically binds to a KRLG polypeptide (e.g., a human KRLG polypeptide), an antibody that specifically binds to a CD8 polypeptide (e.g., a human CD8 polypeptide), and optionally an antibody that specifically binds to a CD3 polypeptide (e.g., a human CD3 polypeptide) such that flow cytometry can be performed to determine the levels of KLRG1⁺/CD8⁺ T cells within a sample. For example, a kit provided herein can include an anti-human KRLG polypeptide antibody that is fluorescently labelled, an anti-human CD8 polypeptide antibody that is fluorescently labelled, and optionally anti-human CD3 polypeptide antibody that is fluorescently labelled. In general, each antibody of a kit provided herein is attached (e.g., covalently attached) to a different fluorescent label. Any appropriate fluorophore can be attached to the antibodies of a kit provided herein. Examples of such fluorophores include, without limitation, fluorescein isothiocyanate, phycoerythrin, allophycocyanin, and peridinin chlorophyll protein.

In some cases, a kit provided herein can include an antibody that specifically binds to a CD57 polypeptide (e.g., a human CD57 polypeptide). For example, a kit provided herein can include an anti-human KRLG polypeptide antibody that is fluorescently labelled, an anti-human CD8 polypeptide antibody that is fluorescently labelled, an anti-human CD3 polypeptide antibody that is fluorescently labelled, and an anti-human CD57 polypeptide antibody that is fluorescently labelled.

In some cases, a kit provided herein can include a dye that stains dead cells or cells with comprised plasma membranes to identify viable cells. For example, a kit provided herein can include an anti-human KRLG polypeptide antibody that is fluorescently labelled, an anti-human CD8 polypeptide antibody that is fluorescently labelled, an anti-human CD3 polypeptide antibody that is fluorescently labelled, and one or more dead cell exclusion dye(s). Examples of such dyes include, without limitation, propidium iodide, 7-aminoactinomycin D (7-AAD), DAPI, Monomeric Cyanine Nucleic Acid Stains (Thermo Fisher Scientific), Sytox® Dead Cell Stains (Thermo Fisher Scientific), LIVE/DEAD® Fixable Dead Cell Stains (Thermo Fisher Scientific), and Zombie Dyes (Biolegend).

In some cases, a kit provided herein can include a red blood cell lysis buffer designed to lyse red blood cells with minimal effects on leukocytes. Such a buffer can be included to allow for the use of whole blood. In general, a red blood cell lysis buffer includes ammonium chloride, potassium carbonate, and EDTA.

Other optional ingredients that can be included within a kit provided herein include, without limitation, positive control samples and negative control samples. A positive control sample can be a sample of PBMCs containing a known amount of KLRG1⁺/CD8⁺ T cells. In some cases, a kit provided herein can contain of set of positive control samples (e.g., two, three, four, five, or more samples) with each positive control sample containing a higher amount KLRG1⁺/CD8⁺ T cells. A negative control sample can be a sample of PBMCs that lacks KLRG1⁺/CD8⁺ T cells.

In some cases, a kit provided herein can include instructions or a package insert setting forth one or more cut off values. For example, a kit provided herein can include instructions or a package insert setting forth a cut off value for a moderately elevated level of KLRG1⁺/CD8⁺ T cells as described herein. In some cases, a kit provided herein can include instructions or a package insert setting forth a cut off value for a highly elevated level of KLRG1⁺/CD8⁺ T cells as described herein. In some cases, a kit provided herein can include instructions or a package insert setting forth (a) a cut off value for a moderately elevated level of KLRG1⁺/CD8⁺ T cells as described herein and (b) a cut off value for a highly elevated level of KLRG1⁺/CD8⁺ T cells as described herein.

In some cases, a kit described herein can include each component of the kit within a single package (e.g., a box). For example, a kit provided herein can be one package that includes a first container containing an anti-human KRLG polypeptide antibody that is fluorescently labelled, a second container containing an anti-human CD8 polypeptide antibody that is fluorescently labelled, and optionally a third container containing an anti-human CD3 polypeptide antibody that is fluorescently labelled.

The invention will be further described in the following examples, which do not limit the scope of the invention described in the claims.

### EXAMPLES

### Example 1—Elevated Levels of KLRG1⁺/CD8⁺ T Cells Allows for Viral Control of CMV Infections

### Methods

### Study Participants

Lung transplant recipients (LTRs) who were CMV seronegative and received lung tissue from a CMV seropositive donor are referred to as Donor+/Recipient− LTRs or D⁺R⁻LTRs. Donor+/Recipient− LTRs from the Johns Hopkins Lung Transplant Program were identified (Table 4) and provided informed written consent for participation in a Johns Hopkins Medicine Institutional Review Board-approved protocol. All patients were treated with three-drug immunosuppression (a calcineurin inhibitor (e.g., tacrolimus or cyclosporine), a cell cycle inhibitor (e.g., cellecept, azathioprine, everolimus, etc.) and prednisone). Antiviral prophylaxis with ganciclovir and/or valganciclovir was used for the initial three months after transplant. Patients were prospectively monitored at least weekly for the development of primary CMV infection (defined as de novo detection of viral replication by quantitative PCR (qPCR)). CMV viral loads were determined by qPCR of plasma by the Johns Hopkins Hospital Clinical Virology Laboratory (CMV qPCR). Patients with primary CMV infection were treated with antiviral therapy (ganciclovir and/or valganciclovir) until two consecutive weekly CMV qPCR measurements revealed undetectable viremia. Following completion of antiviral therapy for primary infection, patients continued to be prospectively monitored with CMV PCR at least biweekly, as well as during any symptomatic or clinically indicated time points, for the development of relapsing viremia (defined as the detection of >300 copies/milliliter (mL) of CMV by qPCR from two consecutive samples after the completion of antiviral therapy for primary infection). Patients with relapsing viremia received antiviral therapy (ganciclovir and/or valganciclovir) if clinically indicated. Chronic and acute viremic samples collected at a minimum of 6 months apart (median±SEM, 379±69 days; range 184-1096 days).

TABLE 4

| | | | | Immunosuppression | Primary | | Viral load^{b} |
|---|---|---|---|---|---|---|---|
| | Age | | Primary | at Primary CMV | CMV | Relapsing | (DNA |
| LTR | (y) | Gender | Diagnosis | Onset | Onset^{a} | Viremia | copies/mL) |
| 23 | 50 | M | Idiopathic | CSA 175/100, | 108 | − | 3,310 |
| | | | Pulmonary | RAPA 1¹, Pred 5¹ | | | |
| | | | Fibrosis | | | | |
| 24 | 31 | F | Cystic Fibrosis | TAC 1.5², MMF | 96 | + | 51,100 |
| | | | | 0.5³, Pred 10¹ | | | |
| 25 | 34 | F | Primary | TAC 4², MMF | 129 | − | 16,900 |
| | | | Pulmonary | 0.5², Pred 10¹ | | | |
| | | | Hypertension | | | | |
| 28 | 33 | F | Cystic Fibrosis | TAC 6², MMF | 210 | − | 1,260 |
| | | | | 0.5², Pred 10¹ | | | |
| 29 | 62 | F | COPD | TAC 2.5², MMF | 168 | + | 47,700 |
| | | | | 0.5³, Pred 10¹ | | | |
| 31 | 55 | M | Cystic Fibrosis | TAC 3², AZA | 248 | − | 3930 |
| | | | | 50¹, Pred 5¹ | | | |
| 33 | 51 | F | Idiopathic | TAC 2², MMF | 186 | + | 1,090 |
| | | | Pulmonary | 0.5³, Pred 7.5¹ | | | |
| | | | Fibrosis | | | | |
| 34 | 59 | F | COPD | TAC 4², MMF | 174 | − | 2,710 |
| | | | | 0.25², Pred 10¹ | | | |
| 35 | 27 | M | Cystic Fibrosis | TAC 1², MMF | 219 | − | 1,258 |
| | | | | 0.5², Pred 7.5¹ | | | |
| 36 | 49 | F | Idiopathic | TAC 4², MMF | 167 | + | 9,070 |
| | | | Pulmonary | 0.25², Pred 10¹ | | | |
| | | | Fibrosis | | | | |
| 37 | 56 | F | Obliterative | TAC 5², MMF | 133 | − | 95,400 |
| | | | Bronchiolitis | 0.5⁴, Pred 10¹ | | | |
| 38 | 56 | M | COPD | TAC 1², MMF | 155 | − | 2,010 |
| | | | | 1², Pred 10¹ | | | |
| 40 | 54 | F | COPD | TAC 1.5², MMF | 122 | − | 32,400 |
| | | | | 0.5⁴, Pred 15¹ | | | |
| 41 | 64 | F | Bronchiectasis | TAC 4², MMF | 184 | − | 23,500 |
| | | | | 0.5³, Pred 5¹ | | | |
| 43 | 51 | M | Sarcoidosis | TAC 4², MMF | 83 | + | 1,490 |
| | | | | 0.5², Pred 15¹ | | | |
| 45 | 21 | M | Cystic Fibrosis | TAC 2.5², MMF | 92 | − | 4,840 |
| | | | | 0.5³, Pred 15¹ | | | |
| 46 | 59 | M | COPD | TAC 1.5², MMF | 37 | + | 49,500 |
| | | | | 1², Pred 20¹ | | | |
| 48 | 47 | M | Idiopathic | TAC 2², MMF | 162 | + | 27,331 |
| | | | Pulmonary | 0.25², Pred 10¹ | | | |
| | | | Fibrosis | | | | |
| 51 | 41 | F | Pulmonary | TAC 2², MMF | 214 | − | 1,675 |
| | | | Hypertension | 0.5², Pred 7.5¹ | | | |
| 53 | 35 | F | Cystic Fibrosis | TAC 2², MMF | 125 | + | 47,913 |
| | | | | 0.5², Pred 10¹ | | | |
| 56 | 61 | F | COPD | TAC 1², MMF | 142 | + | 5,997 |
| | | | | 0.5², Pred 10¹ | | | |
| 57 | 56 | M | Idiopathic | TAC 2², MMF | 118 | + | 2,307 |
| | | | Pulmonary | 0.25², Pred 10¹ | | | |
| | | | Fibrosis | | | | |
| 60 | 62 | M | Idiopathic | TAC 4², MMF | 410 | − | 1,465 |
| | | | Pulmonary | 0.25², Pred 10¹ | | | |
| | | | Fibrosis | | | | |
| Summary^{c} | 48.4 (12.5) | M = | | | 160.1 (73.7) | 43.40% | 18,966 (25,227) |
| | | 43.4% | | | | | |

^{a}= Days post-transplant.
^{b}= Viral load at time of sampling
^{c}= Values represent mean or percent of indicated group (SD)
AZA, azathioprine (dose in milligrams); COPD, chronic obstructive pulmonary disease; CSA, cyclosporine (dose in milligrams); F, female; M, male; MMF, mycophenolate mofetil (dose in grams); Pred, prednisone (dose in milligrams); TAC, tacrolimus (dose in milligrams); superscript number = times per day.

### Preparation of Peripheral Blood and Lung Mucosal Mononuclear Cells (PBMCs and LMMCs)

Blood samples and bronchoalveolar lavage (BAL) samples from LTRs were obtained prior to the discontinuation of initial antiviral prophylaxis (referred to as “pre-CMV”) and within 5-14 days of detection of de novo viremia (referred to as “primary CMV”). Peripheral blood mononuclear cells (PBMCs) were isolated from heparinized blood samples by density gradient centrifugation using Ficoll-Paque (GE Healthcare). Study participants underwent standard BAL with instillation of 180 mL of sterile saline in the right middle lobe of the lung. PBMCs and BAL specimens were obtained on the same day. Lung mucosal mononuclear cells (LMMCs) were obtained via centrifugation of BAL fluid. All patients had therapeutic levels of calcineurin inhibitors at the time of sampling. Cells were then washed with PBS, aliquoted at 1×10⁶ cells in 1 mL of freezing medium (Invitrogen) and frozen in liquid nitrogen. PBMCs were thawed rapidly in the presence of 2 units per mL of Benzonase® (EMD Millipore).

### MHC Class I Dextramer and Surface Staining of PBMCs and BALMCs

Frozen PBMCs and BLAMCs were thawed in the presence of 2 units per mL of Benzonase® (EMD Millipore), and approximately 2×10⁶ cells were labeled with Fixable Viability (FV)-510 stain (BD Biosciences) in IMDM medium (GIBCO) for 10 minutes at 37° C. Following incubation, cells were washed with complete medium and stained with the MHC class I Dextramer (Immudex, Copenhagen, Denmark) against known immunodominant CMV epitopes (Table 5) at 25° C. for 25 minutes. Cells were then washed and incubated with a cocktail of conjugated monoclonal antibodies for 30 minutes at 4° C. in phosphate-buffered saline (PBS) with a fish gelatin blocking reagent (Biotium, Fremont, Calif.) and 0.1% sodium azide. After the final wash, cells were fixed with 1% paraformaldehyde (Protein Sciences, Meriden, Conn.) for analysis. A few available pre-transplant PBMC samples (Pre-PBMC) were stained with matching MHC class I Dextramers. Frequencies of CD3⁺CD8⁺ Dextramer positive cells were between 0.00% to 0.01%.

TABLE 5

| HLA Class 1 | Peptide | CMV antigen |
|---|---|---|
| A*1101 | VTEHDTLLY | pp50 |
| A*0201 | NLVPMVATV | pp65 |
| B*0702 | TPRVTGGGAM | pp65 |
| B*0801 | ELRRKMMYM | IE-1 |

### Flow Cytometry

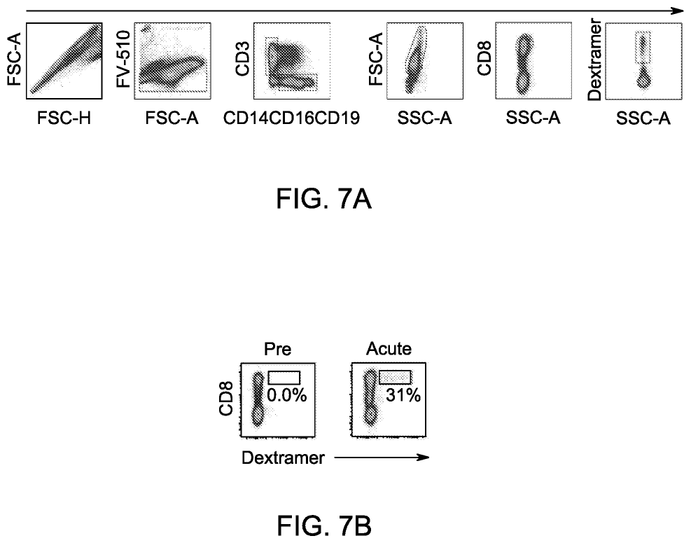

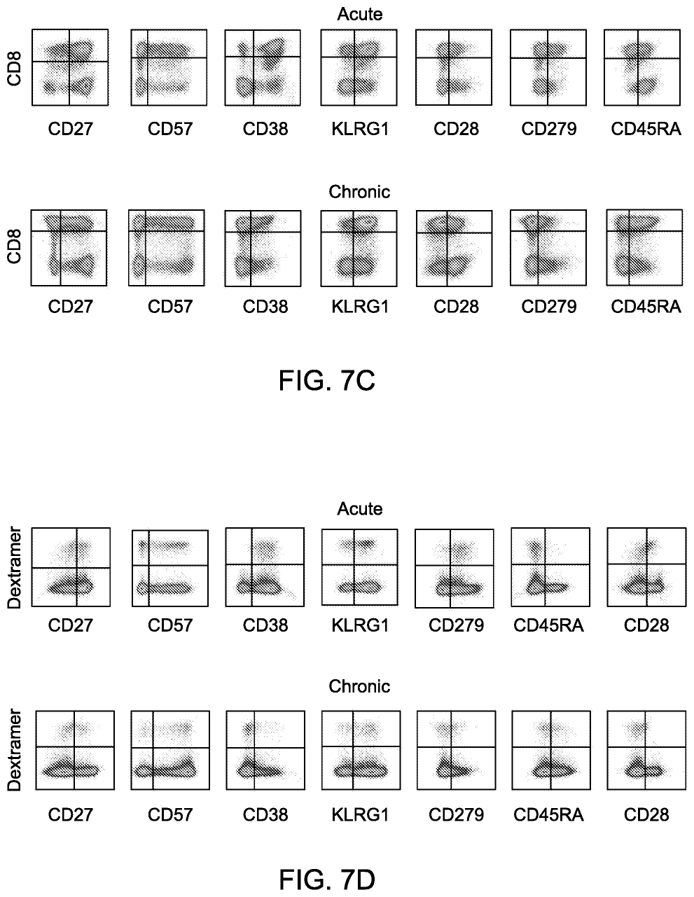

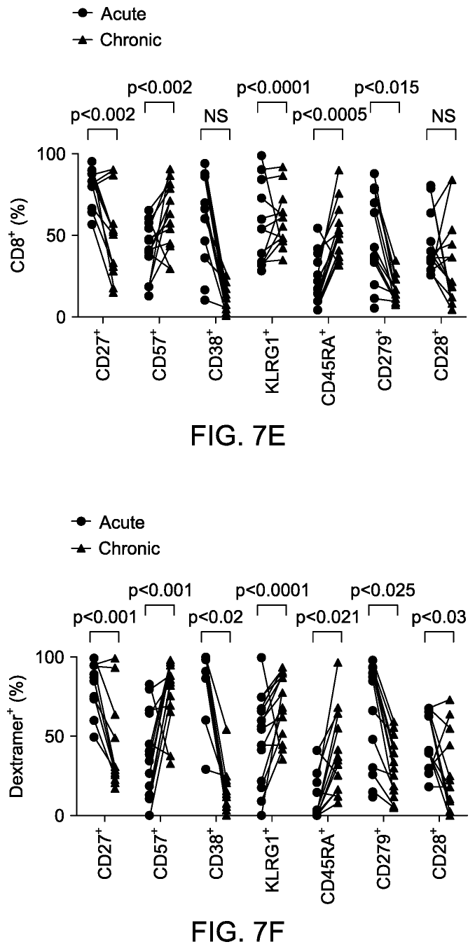

Stained PBMCs were analyzed on a BD Fortessa SORP high-throughput system (HTS). Prior to acquisition, CountBright Beads (Invitrogen) were added to each well. A fixed volume of the stained sample was acquired by HTS. For visualization of ex vivo stained Total and CMV-specific CD8⁺ T cells (FIG. 7A), a compounded gating scheme was used as described elsewhere (Popescu et al., J. Immunol., 193(11):5709-5722 (2014)). The gating strategy was as follows: doublet exclusion based on FSC-A and FSC-H plot, dead cell exclusion based on FV-510 and FSC-A plot, inclusion of CD3⁺ cells based on a CD3⁺ gate with a CD14⁺CD16⁺CD19⁺ exclusion gate (e.g., a CD3-Alexa-700 and a CD14/CD16/CD19-QDot605 plot), and a lymphocyte gate on an FS and SS log plot. CMV-Specific CD8⁺ T cells were gated further on a MHC Class I Tetramer/Dextramer⁺ population (e.g., a MHC Class I Dextramer-PE and SSC-A plot). Data analysis and graphic representations were completed using FlowJo v. 10 (TreeStar, Ashland, Ore.).

### In Vitro Short-Term Stimulation and Intracellular Cytokine Staining (ICS)

Single pools of overlapping 15-mer peptides for pp65 (JPT, Berlin, Germany) were used. PBMCs were cultured in round-bottom tissue culture tubes in the presence or absence of pooled pp65 peptides (1 μg/mL). All stimulations for intracellular cytokine production were performed using 1×10⁶ cells per condition for 6 hours at 37° C. with brefeldin-A (10 μg/mL) (Sigma) added for the final 4 hours of culture. When measuring CD107a, Monensin (5 μg/mL) along with brefeldin-A and an anti-CD107a-Pacific Blue were added at the beginning of the culture. All cells were collected for flow cytometric analysis with a range of 0.5-1×10⁶ total events collected per condition. All gates for cytokine frequencies were set using the medium alone control and subtracted from peptide re-stimulated samples frequencies.

### In Vitro Antigen-Specific T Cell Proliferation

Thawed frozen PBMCs were labeled with CytoTell Green (AAT Bioquest) or 0.2 μM CFSEDA (Invitrogen, Carlsbad, Calif.) in IMDM medium (GIBCO, Grand Island, N.Y.) for 10 minutes at 37° C. Cells were washed and plated with IMDM supplemented with 10% fetal calf serum (FCS) in a 96-well plate. For antigen-specific stimulation, PBMCs were stimulated in the presence of 2 μM of single CMV pp65 peptides (Table 5) or a pool of pp65 peptides for 6 days and then harvested and prepared for flow cytometry.

In some experiments, proliferating cells were re-stimulated in vitro with the CMV pp65 peptide mix as indicated above. Briefly, cells were harvested at day 6, washed, rested overnight in medium alone, and stimulated for a time in the presence or absence of the CMV pp65 peptide mix for 6 hours (according to primary cultures were pulsed/un-pulsed with peptide). In some instances, primary cultures were pulsed (e.g., repeated exposure and removal of peptides). PBMCs were assessed for proliferation using simultaneous CFSE (Carboxyfluorescein succinimidyl ester) dilution and cytokine production measured by intracellular cytokine staining (ICS). Based on the Boolean gating analysis, cytokine co-expression was determined using SPICE.

### Chromatin Immuno-Precipitation (ChIP) Assay

ChIP was performed using a Zymo-Spin™ ChiP kit according to manufacturer's recommendation with modifications. Briefly, 5-6×10⁶ cells were fixed in 1% paraformaldehyde for 10 minutes at 37° C. 2.5 M glycine was added to stop the fixation, and nuclei were extracted using centrifugation. Extracted nuclei, containing the chromatin, were digested using Atlantis MNase (Zymo Research). Following digestion, isolated nuclei were sonicated to release digested chromatin from the nuclei. Digested chromatin was then incubated with the ChIP-grade mouse monoclonal antibody against human TBX21 (“TBET”) (Clone 39D, Santa Cruz) or the control mouse IgG antibody (Santa Cruz) overnight at 4° C. Following overnight incubation, the antibody-bound chromatin was immunoprecipitated by MagnaChIP Protein A+G magnetic beads (EMD Millipore). After a series of washes, bound DNA was isolated and stored at −80° C. PCR was performed with sets of primers (Table 6) designed to amplify either promoter regions or control regions of the genes of interest.

TABLE 6

| Target | Forward | Reverse |
|---|---|---|
| IFN-γ | TACCAGGGCGAAGTGGG | CACCTGTGCCATTCTGG |
| | GAGG | TGGG |
| | (SEQ ID NO: 1) | (SEQ ID NO: 2) |
| IL-4 | AATAGGTGTCGATTTGC | CCAAGTGACTGACAATC |
| | AGTGACAATGTG | TGGTCTAACGAA |
| | (SEQ ID NO: 3) | (SEQ ID NO: 4) |
| GAPD | TCCTTCTGTTTCATCCA | TACTAGCGGTTTTACGG |
| H | AGC | GCG |
| | (SEQ ID NO: 5) | (SEQ ID NO: 6) |
| KLRG | AGCATCTACAGTGTCAT | GATTCAAATTTGGGGGC |
| 1 | GGGG | TGCCT |
| | (SEQ ID NO: 7) | (SEQ ID NO: 8) |

### Transcription Factor Binding Site Prediction

For prediction of TBX21 DNA binding sites on the KLRG1 promoter, a Bioconductor R package called TFBSTools was used. TFBSTools used the JASPAR 2016 transcription factor DNA binding motif database to scan through the promoter region of KLRG1 (e.g., 1272 bp upstream of the first translation initiation ATG (8988365:8989636 GRCh38.p 7) (ENST00000356986.7)) looking for TBX21 DNA binding motifs. Default parameters of searchSeq were used for scanning potential TBX21 DNA binding sites except that a min·score parameter (e.g., minimum score as calculated by searchSeq) was set to 80%.

### Statistical Analysis and Data Visualization

Statistical analysis was performed by using GraphPad Prism 7 (GraphPad, La Jolla, Calif.), JMP12 (SAS, Cary, N.C.), and R (Team, 2016). Mann-Whitney-Wilcoxon and Spearman's rank correlations were used, and a two-tailed P value of less than 0.05 was considered statistically significant.

### Results

Patient Characteristics and Clinical Phenotypes During and Following Primary CMV Infection in D⁺R⁻LTRs

This study evaluated the acute CMV-specific CD8⁺ T_{AEFF} cell phenotype and functional responses in a cohort of 23 D⁺R⁻LTRs during acute primary CMV infection and followed a subset as primary CMV transitioned into chronic CMV infection. The clinical characteristics of these patients during acute/primary de novo viremia are shown in Table 4. Using close prospective monitoring, primary CMV infection was detected at a median of 165 days post-transplant following discontinuation of CMV prophylaxis therapy. Prospective, standard of care monitoring in this high-risk population continued and detected relapsing viremia in 11 of 23 LTRs (relapsers) within the first 6 months of chronic infection in contrast to 12 of 23 LTRs that demonstrated immune control (controllers) following discontinuation of antiviral therapy for acute/primary CMV infection. All episodes of relapsing viremia occurred independent of acute rejection episodes, augmented immunosuppression, or active infections. Additionally, there was no clinical evidence of ganciclovir-resistant CMV in any of the LTR relapsers in this cohort.

CD57 and KLRG1 are Rapidly Induced in CMV-Specific Acute CD8⁺ T_{AEFF} Cells (CD27⁺) During Primary Infection and Undergo Phenotypic Progression to TEM (CD27⁻) into Chronic Infection

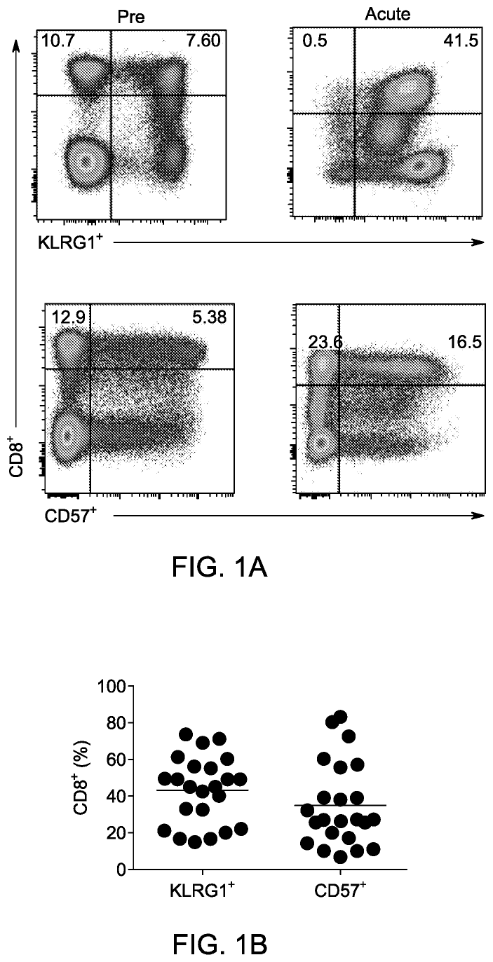

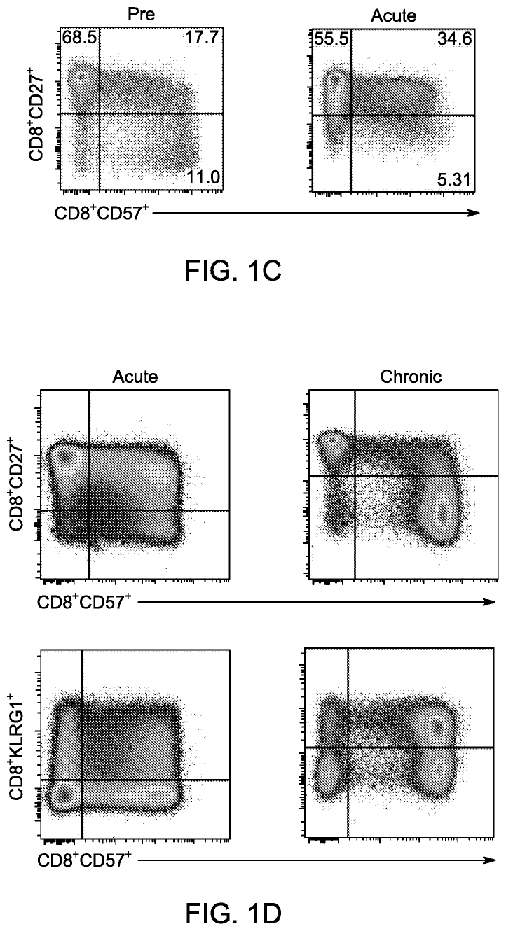

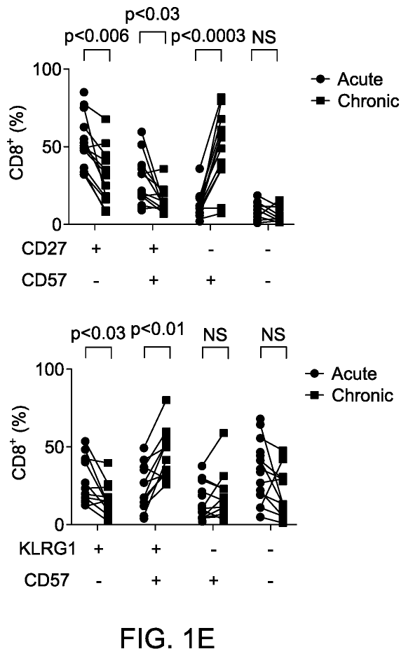

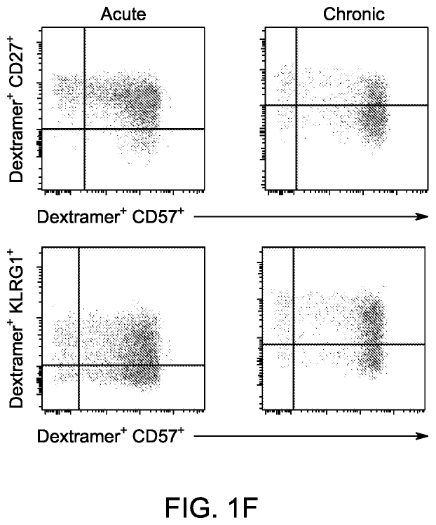

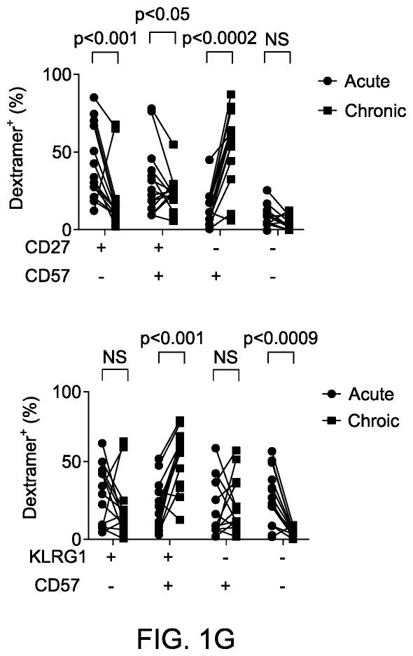

Next, the dynamics of CD57 and KLRG1 expression, along with other major phenotypic markers, were assessed in circulating Total CD8⁺ T cells and CMV-specific acute CD8⁺ T_{AEFF} cells in the D⁺/R⁻LTRs cohort during primary CMV infection. Increased frequencies of CD57⁺, KLRG1⁺, and CD57⁺CD27⁺ total CD8⁺ T cells were observed during acute/primary CMV infection (FIG. 1A) when compared to pre-CMV infection levels (FIGS. 1A-C). Determination of CD57 expression in relation to KLRG1 and CD27 expression found an enrichment of Total CD8⁺CD57⁺ (FIGS. 1D and E) and CMV-specific CD8⁺CD57⁺ Dextramer⁺ T_{AEFF} cells in the CD27⁺ subset (FIGS. 1F and 1G) during acute infection with evidence of progression to a CD57⁺CD27⁻ subset indicative of chronic infection. Similarly, the proportion of CD57⁺KLRG1⁺ cells increased in Total CD8⁺ T cells (FIGS. 1D and E) and CMV-specific CD8⁺CD57⁺KLRG1⁺ Dextramer⁺ T_{AEFF}-cells (FIGS. 1F and 1G) during progression from primary CMV into chronic CMV infection. Assessment of other phenotypic markers including CD28, CD38, CD45RA and CD279 (programmed death-1; PD-1) were also made during primary CMV infection and chronic CMV infection. During primary infection, both Total and CMV-specific T_{AEFF} T cells predominantly expressed a CD27^{hi}CD28^{+/-}CD38⁺CD279⁺CD45RA⁻ acute T effector phenotype and progressed to a CD27^{lo/-}CD28⁻CD38⁻CD279⁻CD45RA^{+/hi} T effector memory (T_{EM}) phenotype in chronic infection (FIGS. 7E and 7G). Together, these results demonstrate an unanticipated rapid induction of CMV-specific CD8⁺CD57⁺ T_{AEFF}-cells and CD8⁺KLRG1⁺ T_{AEFF} during acute/primary CMV infection, with a subset of T_{AEFF} that co-express these markers in conjunction with CD27. These effector populations became progressively enriched during progression into chronic infection, bearing a classical T_{EM} phenotype.

KLRG1⁺ and CD57⁺ CMV-Specific CD8⁺ T_{AEFF} Demonstrate Proliferative Capacities

To address the stability of CD57 and KLRG1 expression in CMV-specific CD8⁺ T cells, expression for each polypeptide was assessed in three patients during primary CMV infection, following clearance of CMV infection (contraction phase), and during chronic infection (6-9 months later). Despite contraction of CMV-specific CD8⁺ Dextramer⁺ frequencies during the contraction phase, and into chronic infection, expression of both CD57 and KLRG1 remained stable or increased. Consistent with an enrichment of KLRG1⁺ and CD57⁺ T cells into the T_{EM} stage, often associated with chronic infection, CD27, a marker for T_{AEFF} cell, expression was reduced. Recent studies have shown the involvement of CMV-specific CD8⁺ T cell proliferation during primary infection for the establishment of immune viral control and for the up-regulation of the major Type-1 transcription factor, T-BET, in high-risk LTRs (Pipeling et al., J. Infect. Dis., 204(11):1663-71 (2011); Popescu et al., J. Immunol., 193(11):5709-5722 (2014); and Popescu et al., J. Immunol., 196(2):877-90 (2016)).

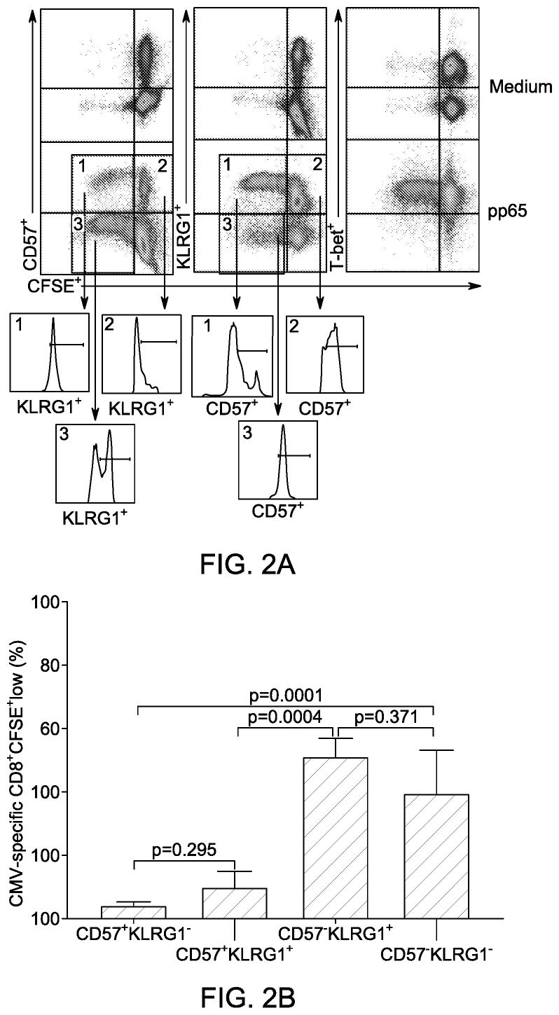

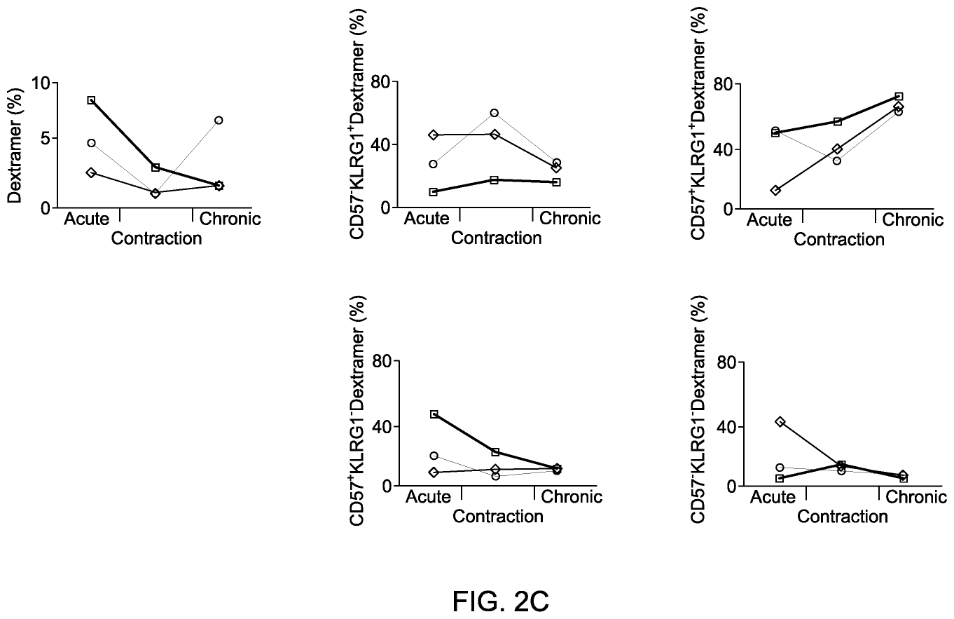

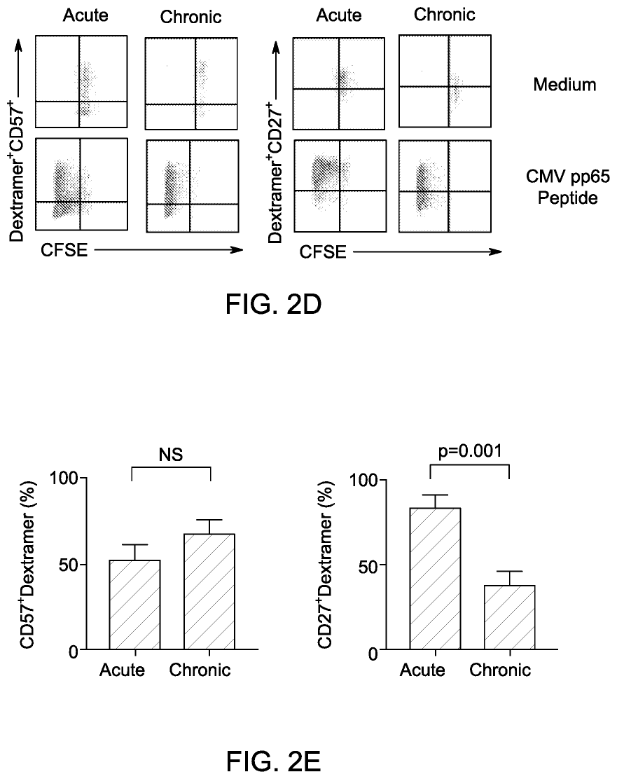

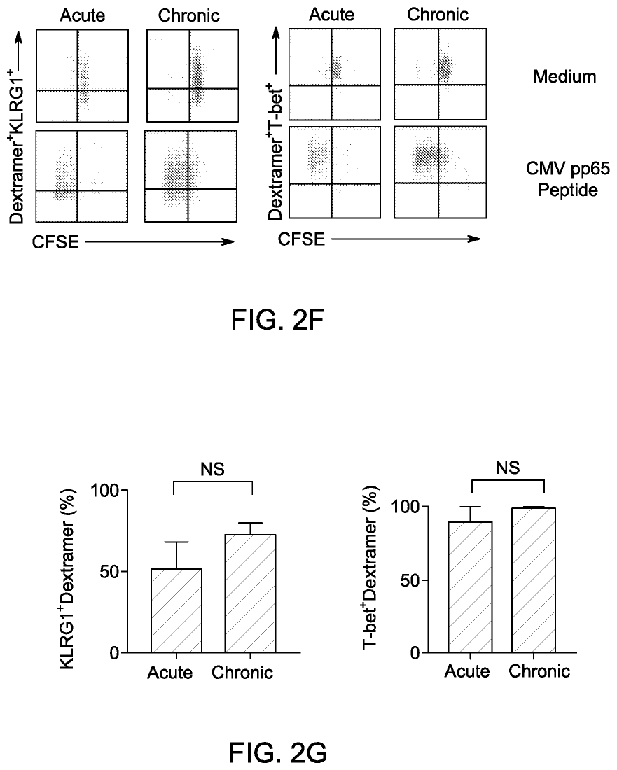

CMV-specific CD8⁺ T cells expressing KLRG1 or CD57 were assessed for the capacity to proliferate in response to CMV peptides in vitro. Using an overlapping 15-mer peptide pool of the major CMV antigen, phosphoprotein 65 (pp65), or single HLA-restricted cognate peptides recognized by respective CMV Dextramers, measurements of in vitro CMV-specific proliferation were made using CFSE dilution assays at 6 days in the same three patients (e.g., during primary CMV infection, during contraction phase, and during chronic infection). KLRG1⁺ CD8⁺ T cells from PBMC samples proliferated at levels similar to T-BET⁺ CD8⁺ T cells (FIG. 2B). While CMV-specific CD8⁺CD57⁺ T cells proliferated at a lower level compared to KLRG1⁺ or T-BET⁺ CD8⁺ T cells, this was still significant (FIGS. 2B, 2C). In addition, the majority of KLRG1⁺ proliferating cells expressed CD57⁺ and vice versa (FIG. 2B insets). To further investigate the proliferative capacity of CMV-specific CD8⁺CD57⁺ Dextramer⁺ T cells, assessments of proliferation were made on day 6 during primary CMV infection using only the CMV-specific cognate peptide for each Dextramer for re-stimulation. Under these conditions, robust proliferation was observed in CMV-specific CD8⁺ Dextramer⁺ T cells (FIG. 2D, 2E). Comparison of acute and chronic proliferative responses in CMV-specific CD8⁺ Dextramer⁺ T cells in response to dextramer cognate peptides for CD57, KLRG1, and T-BET showed similar proliferative capacities for each population in acute versus chronic CMV infection. Collectively, these results demonstrate that KLRG1⁺ and CD57⁺ CMV-specific CD8⁺ T cells exhibit in vitro proliferative capacities and that KLRG1⁺ and CD57⁺ were enriched as CMV infection progressed from primary to chronic CMV infection.

KLRG1 Expression, but not CD57, Correlates with CMV-Specific CD8⁺ T_{AEFF} Cell Multifunction

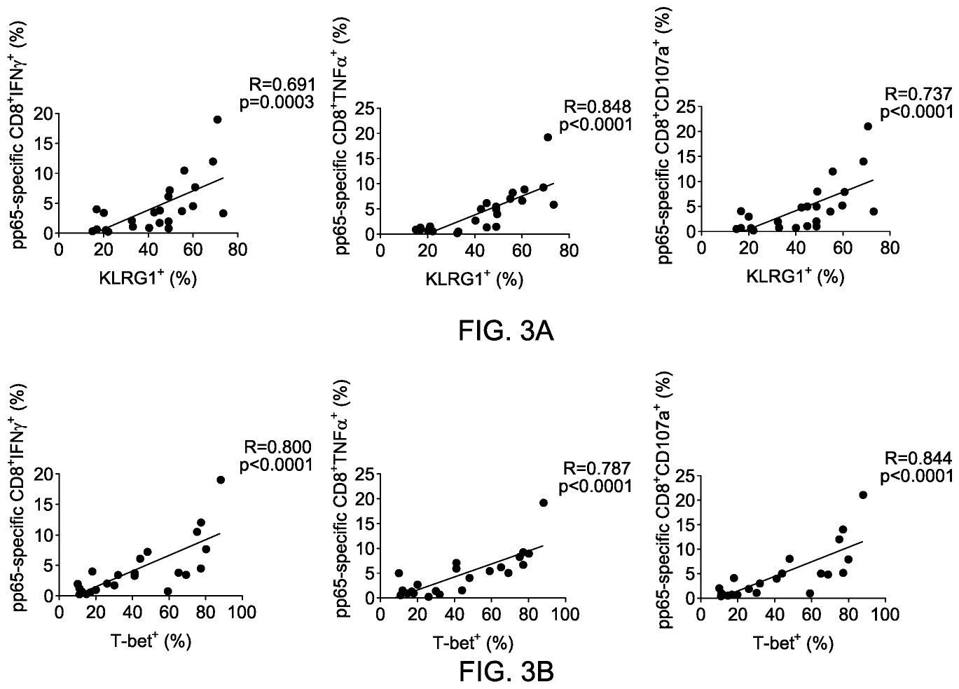

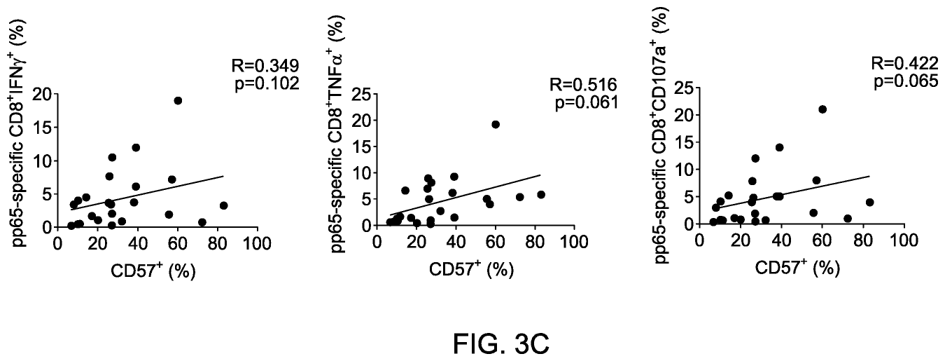

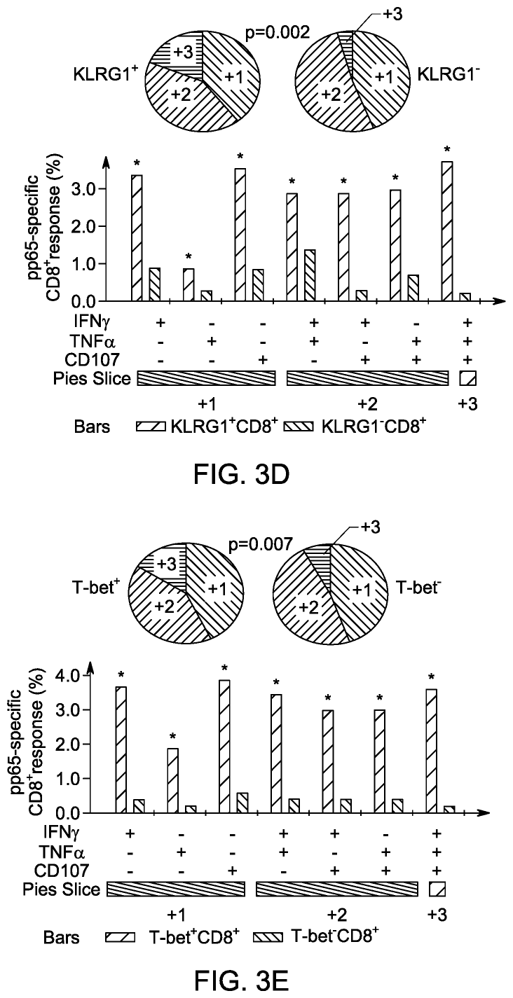

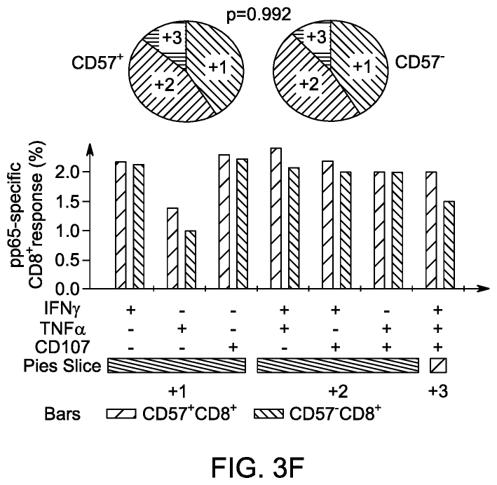

Next, assessments were made regarding whether KLRG1 and/or CD57 surface expression were predictive of CMV-specific CD8⁺ effector function during acute primary CMV infection. Comparisons were made between KLRG1 and/or CD57 predictive power and T-BET's correlation to T cell effector function. Using pp65 pooled peptides, ex vivo CMV-specific IFN-γ, TNF-α, and CD107a expression were measured by flow cytometric intracellular staining (ICS) in a 6 hour re-stimulation assay. Here, KLRG1⁺ expression was correlated with CMV-specific IFN-γ, TNF-α, and CD107a T_{AEFF} frequencies, similar to T-BET⁺ expression in CD8⁺ T cells (FIGS. 3A, 3B). By comparison, CD57⁺ expression was an inferior functional correlate for CMV-specific T_{AEFF} responses and did not reach statistical significance (FIG. 3C). Next, assessments were made regarding CMV-specific CD8⁺ T_{AEFF} multifunction (e.g., the capacity to produce more than one effector molecule) using Boolean analysis. KLRG1⁺CD8⁺ T_{AEFF} cells demonstrated significantly increased CMV-specific effector multifunction (IFN-γ, TNF-α, and CD107a) compared to KLRG1⁻CD8⁺ T_{AEFF} cells (FIG. 3D). Similar findings were observed when T-BET⁺ versus T-BET⁻ CMV-specific effector responses were compared (FIG. 3E). However, in contrast, CMV-specific T_{AEFF} frequencies and multifunction were similar between CD57⁺ and CD57⁻CD8⁺ T cells (FIG. 3F). Taken together, these results demonstrate that KLRG1 expression is a significant functional correlate for both individual and multifunctional CMV-specific CD8⁺ T_{AEFF} responses, whereas CD57 expression is a relatively poor predictor of effector function.

KLRG1 Expression Correlates with T-BET Expression and T-BET Associates with the Human KLRG1 Promoter

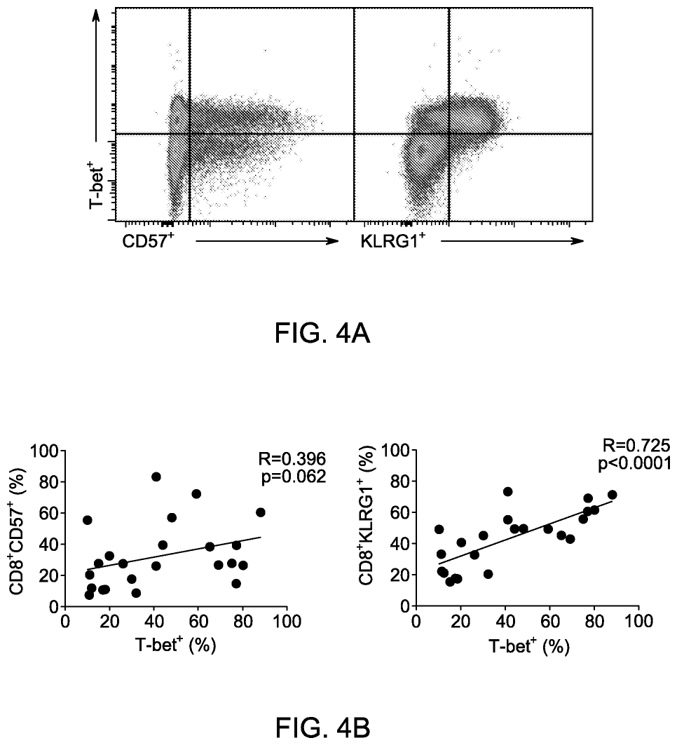

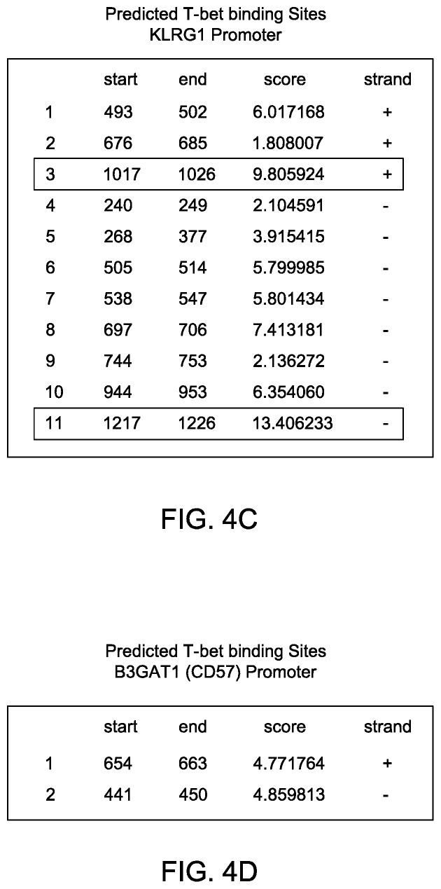

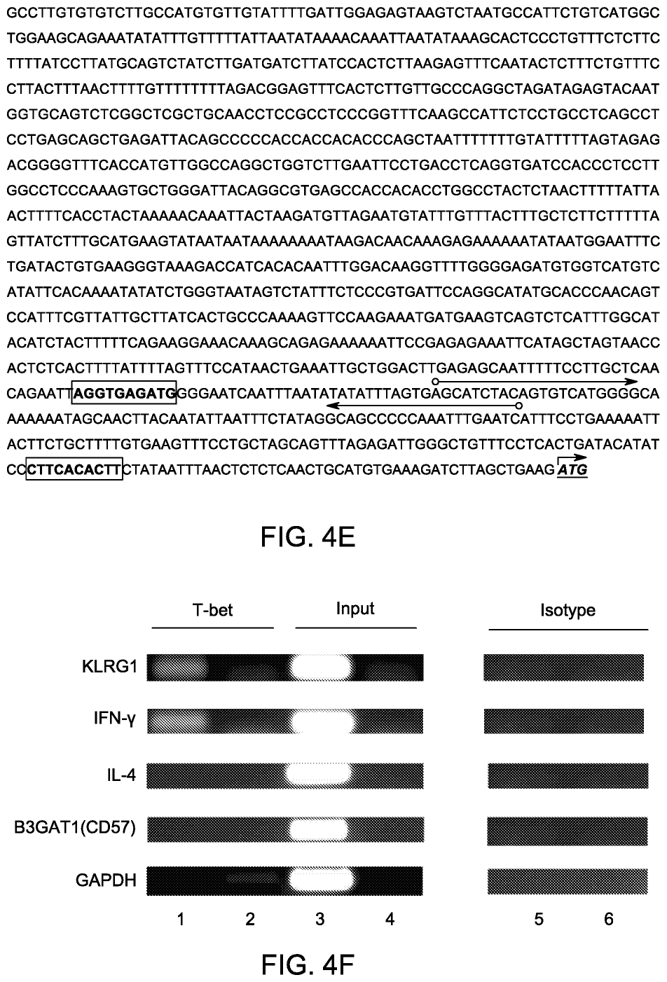

The finding that KLRG1 expression correlated with CMV-specific CD8⁺ T_{AEFF} function prompted investigation into whether there was a causal relationship between T-BET and KLRG1 in human T cells. In the LTR cohort, CD8⁺ T cells showed T-BET and KLRG1 co-expression but little co-expression of either T-BET or KLRG1 with CD57 (FIG. 4A). Using Spearman rho analysis, intracellular T-BET and surface KLRG1 expression were assessed and found to be significantly correlated in CD8⁺ T cells in the LTR cohort during primary CMV infection (FIG. 4B). To further demonstrate a direct association between T-BET and the KLRG1 promoter, putative T-BET binding sites were identified within the promoter region of the KLRG1 gene (FIGS. 4C, 4E), using a consensus T-BET DNA binding matrix and a transcription factor binding site analysis tool (TFBS tools). T-BET binding site with the highest TFBS score (13.4) were comparable to the well-defined T-BET binding site in the IFN-γ promoter (TFBS score=11.6; data not shown). In contrast, putative binding sites were not identified within the CD57 promoter (FIG. 4D). Based on this analysis, a Chromatin Immunoprecipitation assay (ChIP assay) was performed using DNA extracted from PMA/anti-CD3-activated Jurkat T cells, previously shown to have rapid up-regulation of T-BET. As shown in FIG. 4F, T-BET bound to the KLRG1 promoter, indicating a direct regulatory association between T-BET and KLRG1. As expected, the IFN-γ promoter (positive control) was bound by T-BET IP. Also as expected, the IL-4 promoter (negative control), GAPDH promoter (housekeeping), or isotype IgG did not bound by T-BET. Taken together, these results demonstrate a direct regulatory relationship between T-BET and the KLRG1 promoter, supporting a role in the regulation of KLRG1 gene expression.

D⁺R⁻ LTR controllers demonstrate increased total and CMV-specific acute/primary KLRG1⁺CD8⁺ T_{AEFF} Cells, and LTR Relapsers do not

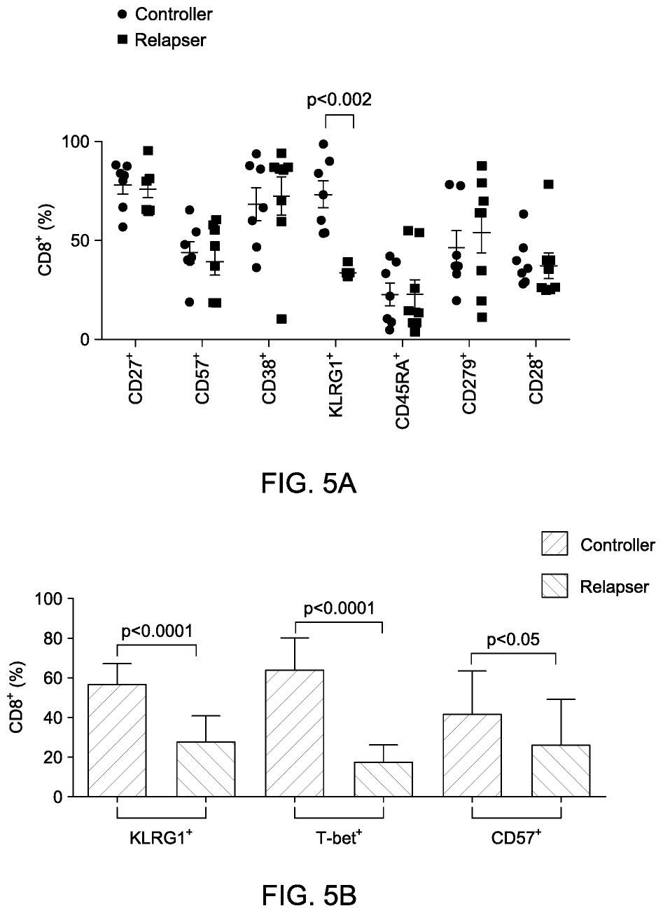

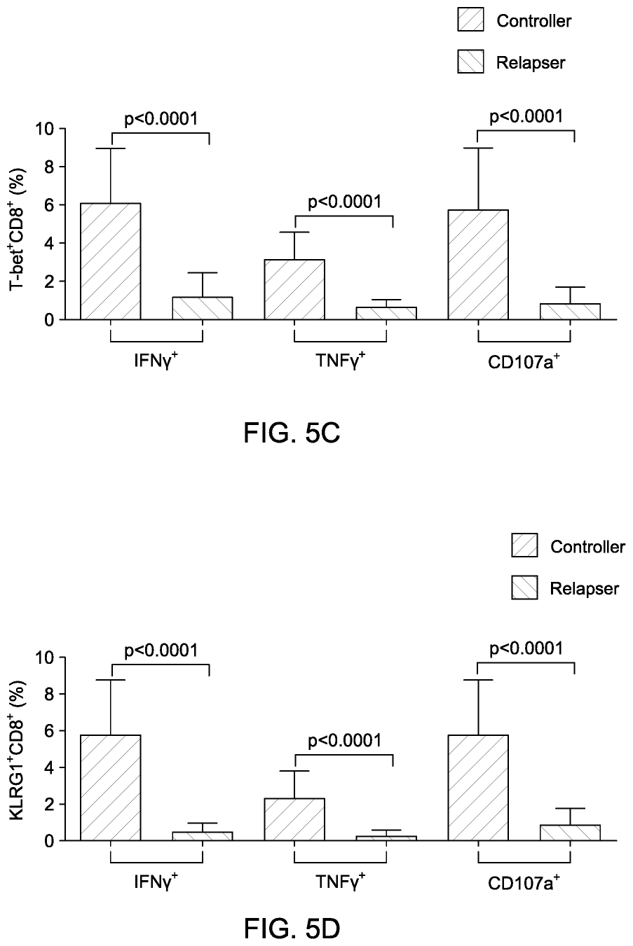

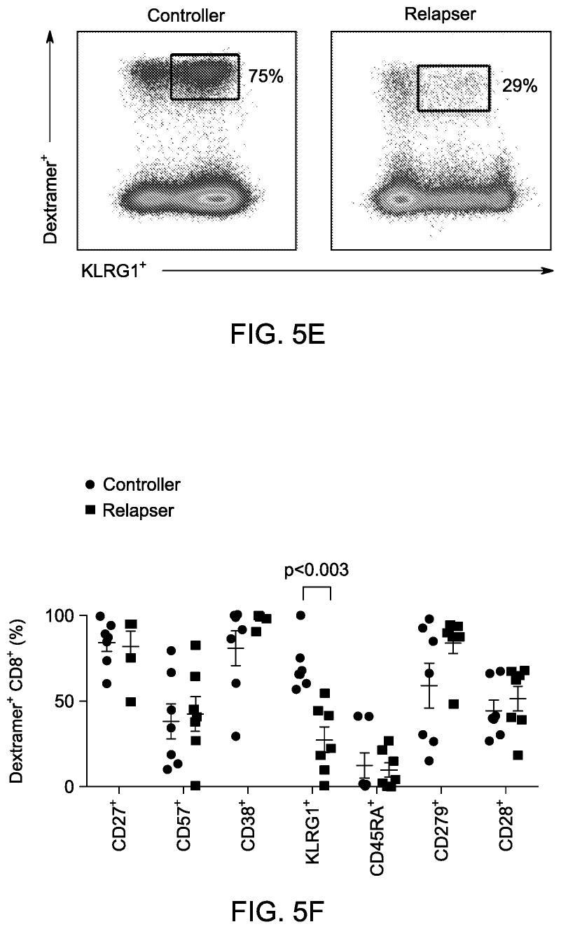

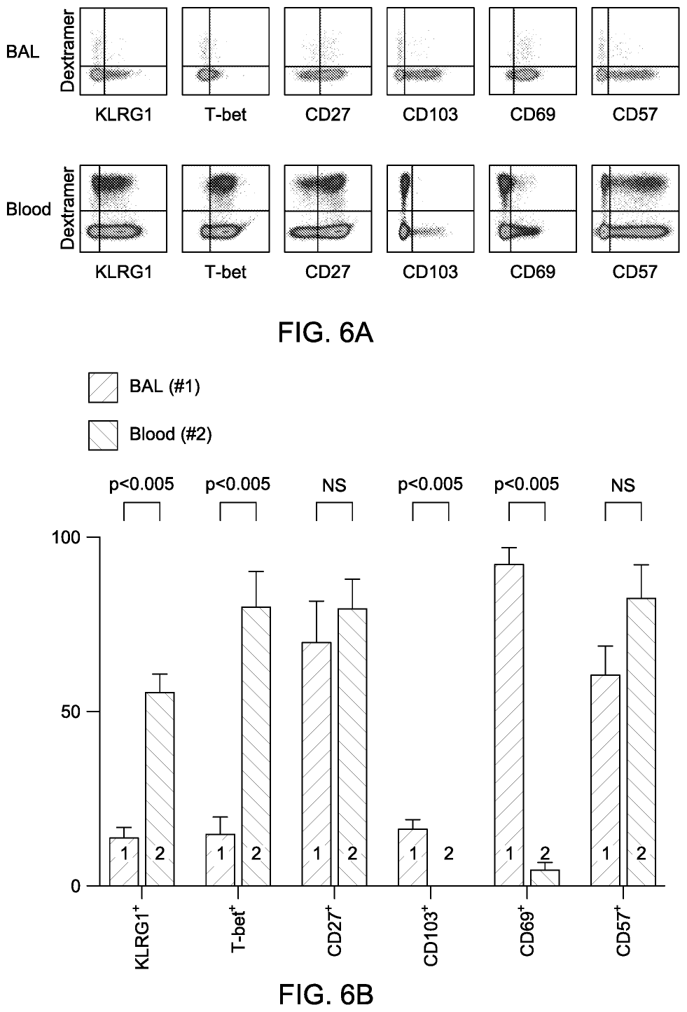

KLRG1 surface expression was assessed for its ability to predict the capacity for D⁺R⁻LTRs to establish early viral control in the first six months following primary CMV infection. Total CD8⁺ T cells form 14 patients were analyzed for the presence of CD27, CD57, CD38, KLRG1, CD45RA, CD279, and CD28. Only KLRG1⁺ expression differentiated CMV infection controllers from CMV infection relapsers (FIG. 5A). CD8⁺ T cells expressing KLRG1⁺ and/or T-BET⁺ populations were enriched in controllers compared to relapsers (FIG. 5B). In addition, CMV pp65-specific KLRG1⁺CD8⁺ T_{AEFF} cells produced significantly more immune effector cell molecules IFN-γ, TNF-α, and CD107a in the controllers compared to relapsers (FIG. 5D). Similarly, CMV pp65-specific T-BET⁺ CD8⁺ T_{AEFF} cells also produced significantly more immune effector cell molecules in the controllers compared to the relapsers (FIG. 5C). Lastly, when looking at KLRG1 expression in CMV Dextramer⁺CD8⁺ T_{AEFF} cells, KLRG1 expression differentiated LTR controllers from relapsers (FIG. 5E, 5F). Collectively, these results demonstrate that KLRG1⁺/CD8⁺ T cells were responsible for the capacity of D⁺R⁻LTRs to establish early immune viral control.

Total and CMV-Specific Lung Mucosal CD8⁺ T_{AEFF} Cells from BAL Express Reduced Levels of KLRG1 and T-BET Compared to the Blood During Acute/Primary CMV Infection

In contrast to circulating CD8⁺ T cells, mucosal CD8⁺ resident memory T cells (T_{RM}) were recently reported to express reduced levels of T-BET (Hombrink et al., Nat. Immunol., 17(12):1467-1478 (2016)). While previous work demonstrated high immune effector function in CMV-specific lung mucosal cells derived from BAL during acute primary CMV infection, little is known about KLRG1 and T-BET expression in lung mucosal CMV-specific CD8⁺ T_{AEFF} cells during acute/primary CMV infection.

Assessments were made regarding KLRG and T-BET expression in lung mucosal versus blood CMV-specific CD8⁺ T_{AEFF} cells during acute primary CMV infection. Both lung Total CD8⁺ T cells and CMV-specific CD8⁺ lung T_{AEFF}-cells expressed reduced levels of both T-BET and KLRG1 as compared to these cells in the blood. In contrast, CD57, CD27, and CD103 expression were similar in Total CD8⁺ T cells and CMV-specific CD8⁺ T_{AEFF} cells between the two compartments. Thus, lung mucosal CMV-specific CD8⁺ T_{AEFF} cells demonstrated a predominant KLRG1^{lo}T-BET^{lo}CD103^{+/-}CD57^{+/-}CD69⁺CD27⁺ phenotype in contrast to systemic CMV-specific CD8⁺ T_{AEFF} cells, which exhibited a predominant KLRG1⁺ T-BET⁺CD103⁻CD57⁺CD69⁻CD27⁺ phenotype. Together, these results demonstrate significant differences in Total and CMV-specific CD8⁺ T_{AEFF} cells in the lung versus the blood during acute/primary CMV infection.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-31. (canceled)

32. A method for treating a CMV infection, wherein said method comprises administering, to a mammal (a) having said CMV infection and (b) identified as lacking an elevated level of KLRG1⁺/CD8⁺ T cells, an antiviral agent.

33. The method of claim 32, wherein the mammal is a human.

34. The method of claim 32, wherein the mammal is a recipient of a transplant.

35. The method of claim 34, wherein said transplant is positive for said CMV infection.

36. The method of claim 34, wherein said mammal is a donor positive/recipient negative mammal for said CMV infection or a donor negative/recipient positive mammal for said CMV infection.

37-39. (canceled)

40. The method of claim 32, wherein said antiviral agent is ganciclovir, valganciclovir, foscarnet, or cidofovir.

41. (canceled)

42. A method for treating a CMV infection, wherein said method comprises:
(a) administering an antiviral agent to a mammal having said CMV infection if said mammal was identified as lacking an elevated level of KLRG1⁺/CD8⁺ T cells, and
(b) monitoring said mammal for an uncontrolled CMV infection if said mammal was identified as having a moderately elevated level or a highly elevated level of KLRG1⁺/CD8⁺ T cells.

43. The method of claim 42, wherein the mammal is a human.

44. The method of claim 42, wherein the mammal is a recipient of a transplant.

45. The method of claim 44, wherein said transplant is positive for said CMV infection.

46. The method of claim 44, wherein said mammal is a donor positive/recipient negative mammal for said CMV infection or a donor negative/recipient positive mammal for said CMV infection.

47. The method of claim 42, wherein said CMV infection is a pre-acute, acute/primary, contraction phase, or chronic CMV infection.

48-51. (canceled)

52. The method of claim 42, wherein said antiviral agent is ganciclovir, valganciclovir, foscarnet, or cidofovir.

53. The method of claim 42, wherein said mammal was identified as lacking said elevated level of KLRG1⁺/CD8⁺ T cells.

54. The method of claim 42, wherein said mammal was identified as having said moderately elevated level of KLRG1⁺/CD8⁺ T cells.

55. The method of claim 42, wherein said mammal was identified as having said highly elevated level of KLRG1⁺/CD8⁺ T cells.

56. A method for monitoring a CMV infection in a manner that avoids the need to administer an antiviral agent to a mammal having said CMV infection, wherein said method comprises assessing a symptom of said CMV infection in said mammal and avoiding administration of an antiviral agent to said mammal, wherein said mammal was identified as having a moderately elevated or highly elevated level of KLRG1⁺/CD8⁺ T cells.

57. The method of claim 56, wherein the mammal is a human.

58. The method of claim 56, wherein the mammal is a recipient of a transplant.

59. The method of claim 58, wherein said transplant is positive for said CMV infection.

60. The method of claim 58, wherein said mammal is a donor positive/recipient negative mammal for said CMV infection or a donor negative/recipient positive mammal for said CMV infection.

61. The method of claim 56, wherein said CMV infection is a pre-acute, acute/primary, contraction phase, or chronic CMV infection.

62-72. (canceled)
